(19) United States
(12) Patent Application Publication
(10) Pub. No.: US 2005/0214750 A1
(43) Pub. Date: Sep. 29, 2005

(54) Compositions and methods for determining the replication capacity of a pathogenic virus

(75) Inventors: Neil T. Parkin, Belmont, CA (US); Colombe Chappey, San Francisco, CA (US); Michael Bates, San Carlos, CA (US)

Correspondence Address: JONES DAY, 222 EAST 41ST ST, NEW YORK, NY, 10017, US

(73) Assignee: ViroLogic, Inc.

(21) Appl. No.: 11/052,741

(22) Filed: Feb. 4, 2005

Related U.S. Application Data
(60) Provisional application No. 60/542,798, filed on Feb. 6, 2004.

(51) Int. Cl. C12Q 1/70; C12Q 1/68; G06F 19/00; G01N 33/48; G01N 33/50

(52) U.S. Cl. 435/5; 435/6; 702/19

(57) ABSTRACT

This invention relates to methods for predicting replication capacity of a virus based on genotype and identifying targets for antiviral therapy by identifying mutations associated with altered replication capacity. The methods are useful, for example, for identifying previously unknown interactions among viral molecules or between viral molecules and host cell molecules that are essential to viral infection and/or replication. By identifying such interactions, novel targets for antiviral therapy can be identified. In another aspect, the invention provides a method for determining that an HIV has an altered replication capacity. In certain embodiments, the method comprises detecting a mutation in a codon of gag that is selected from the group consisting of 437, 439, 441, 442, 454, 478, 479, and 484. In certain embodiments, the mutation is selected from the group consisting of I437L, P439S, E454V, P478L, and I479K In certain embodiments, the mutation is in a codon of gag that is selected from the group consisting of 418, 456, 456, 453, 418, 483, 481, 465, 429, 484, 481, 483, 484, 465, 454, 442, 479, 418, 479, and 486.

This application is entitled to and claims benefit of U.S. Provisional Application No. 60/542,798, filed Feb. 6, 2004, which is hereby incorporated by reference in its entirety.

1. FIELD OF INVENTION

This invention relates, in part, to methods for identifying targets for antiviral drug therapy by assessing correlations between viral genotypes and replication capacity of the virus. The methods are useful, for example, for identifying unrecognized targets for the treatment of viral infections with antiviral drugs. The invention also relates, in part, to methods for determining replication capacity of HIV based upon the HIV's genotype.

### 2. BACKGROUND OF THE INVENTION

More than 60 million people have been infected with the human immunodeficiency virus (“HIV”), the causative agent of acquired immune deficiency syndrome (“AIDS”), since the early 1980s. See Lucas, 2002, Lepr Rev. 73(1):64-71. HIV/AIDS is now the leading cause of death in sub-Saharan Africa, and is the fourth biggest killer worldwide. At the end of 2001, an estimated 40 million people were living with HIV globally. See Norris, 2002, Radiol Technol. 73(4):339-363.

Modern anti-HIV drugs target different stages of the HIV life cycle and a variety of enzymes essential for HIV's replication and/or survival. Amongst the drugs that have so far been approved for AIDS therapy are nucleoside reverse transcriptase inhibitors (“NRTIs”) such as AZT, ddI, ddC, d4T, 3TC, and abacavir; nucleotide reverse transcriptase inhibitors such as tenofovir; non-nucleoside reverse transcriptase inhibitors (“NNRTIs”) such as nevirapine, efavirenz, and delavirdine; protease inhibitors (“PIs”) such as saquinavir, ritonavir, indinavir, nelfinavir, amprenavir, lopinavir and atazanavir; and fusion inhibitors, such as enfuvirtide.

Nonetheless, in the vast majority of subjects none of these antiviral drugs, either alone or in combination, proves effective either to prevent eventual progression of chronic HIV infection to AIDS or to treat acute AIDS. Further, many other viral diseases afflict humans, many of which have no effective therapy to date. Therefore, there remains a need to identify new antiviral compounds in general, and anti-HIV compounds in particular, in order to provide additional options in the treatment of viral diseases. Particularly useful would be methods for identifying anti-HIV compounds that target viral activities other than protease or reverse transcriptase in order to supplement the currently available treatments. The present invention provides methods that address these and other longstanding needs.

### 3. SUMMARY OF THE INVENTION

The present invention provides methods for identifying targets for antiviral therapy. In the methods, targets for antiviral therapy can be identified by determining the location of mutations in the viral genome that affect replication capacity. The change in replication capacity indicates that the genetic loci in which the mutations occur are important for essential viral functions, such as replication and/or infectivity. By identifying the genomic location of the mutations, specific regions of these genes or their encoded gene products can be identified as attractive targets for antiviral therapy.

Thus, in certain aspects, the invention provides a method for identifying a target for antiviral therapy that comprises determining the replication capacity of a statistically significant number of individual viruses, the genotypes of a gene of the statistically significant number of viruses, and a correlation between the replication capacities and the genotypes of the gene, thereby identifying a target for antiviral therapy. The phenotypes of the viruses can be determined according to any method known to one of skill in the art without limitation. Further, the genotypes of the viruses can be determined according to any method known to one of skill in the art without limitation. Finally, a correlation between the phenotypes and the genotype can be determined according to any method known to one of skill in the art, without limitation. Methods for determining such phenotypes, genotypes, and correlations are described extensively below.

In another aspect, the present invention provides methods for predicting a virus's replication capacity based upon the presence of particular mutations in the viral genome. In certain embodiments, the methods are based, in part, on the results of regression analysis of mutations correlated with altered replication capacity as described above. In other embodiments, the methods are based, in part, on the results of univariate analysis of mutations correlated with altered replication capacity. In certain embodiments, the invention provides a method for determining that an HIV has altered replication capacity that comprises detecting a mutation in a codon of gag that is selected from the group consisting of codons 418, 427, 429, 437, 439, 442, 454, 465, 466, 470, 473, 478, 482, 483, 484, and 486. In certain embodiments, the mutation can be selected from the group consisting of K418R, T427P, I437L, P439S, K442G, E454V, F465Y, T470V, T470Y, S473F, P478L, and L486S.

### 4. BRIEF DESCRIPTION OF THE FIGURES

FIGS. 1A and 1B present a diagrammatic representation of a replication capacity assay.

FIG. 2 presents charts demonstrating that replication capacity measurements made using the replication capacity assay are consistent with measurements made using a replication competition assay.

FIGS. 3A and 3B present the distribution of replication capacities identified in 1063 individual wild-type HIV-1 isolates (first data set), and the distribution of replication capacities identified in 544 individual wild-type HIV isolates of subtype B (second data set), respectively.

FIG. 4 presents a scatter plot showing the reproducibility of replication capacity measurements. Each group of circled points represents multiple RC measurements of the same sample.

FIG. 5 presents a depiction of the resistance test vector used in the PHENOSENSE™ assay and its correspondence to the HIV-1 genome.

FIG. 6 presents a table showing mutations in HIV-1 protease and the p6 gag protein (from data set 1) that are associated with high or low replication capacity determined using Fisher's Exact Test, Odds Ratios, and Student's unpaired T-test.

FIGS. 7A and 7B present tables showing mutations in HIV-1 protease, reverse transcriptase, and the p6 gag protein (from data set 2) that are associated with high or low replication capacity determined using Fisher's Exact Test and Student's unpaired T-test, respectively.

FIGS. 8A and 8B present the distribution of replication capacities observed from data set 2 for individual gag mutations associated with increased replication capacity.

FIGS. 9A, 9B, 9C, 9D, 9E, 9F, 9G, and 9H present the distribution of replication capacities observed from data set 2 for individual gag mutations associated with decreased replication capacity.

FIGS. 10A and 10B present the distribution of replication capacities observed from data set 2 for individual RT mutations associated with increased replication capacity.

FIGS. 11A, 11B, 11C, 11D, 11E, and 11F present the distribution of replication capacities observed from data set 2 for individual RT mutations associated with decreased replication capacity.

FIGS. 12A and 12B present the distribution of replication capacities observed from data set 2 for individual PR mutations associated with increased replication capacity.

FIGS. 13A, 13B, 13C, and 13D present the distribution of replication capacities observed from data set 2 for individual PR mutations associated with decreased replication capacity.

FIG. 14 presents an alignment of insertion mutations between codons 458 and 459 of gag that were observed from data set 1, which mutations correlate with altered replication capacity.

FIGS. 15A, 15B, and 15C present an alignment of insertion mutations between codons 452 and 453 or codons 460 and 461 of gag, which mutations marginally correlate with altered replication capacity.

FIG. 16 presents the percentiles of replication capacities in which viruses with particular gag mutations are observed.

FIG. 17 presents a regression tree analysis that diagrams the relative contributions of gag mutations that correlate most strongly with reduced replication capacity. “PT” refers to the length of the insertion near the PTAP domain.

FIG. 18 presents a representation of the distribution of replication capacities observed from a set of viruses isolated from treatment-naïve patients.

FIGS. 19A and 19B present tables showing associations between mutations (FIG. 19A) or length of insertion following the PTAP motif (FIG. 19B) and increased or decreased replication capacity.

FIGS. 20A, 20B, 20C, and 20D present the distribution of replication capacities observed from set of viruses isolated from treatment-naïve patients for individual Gag mutations associated with decreased replication capacity.

FIGS. 21A and 21B present the distribution of replication capacities observed from set of viruses isolated from treatment-naïve patients for individual Gag mutations associated with increased replication capacity.

FIGS. 22A and 22B present the distribution of replication capacities observed from set of viruses isolated from treatment-naïve patients for individual Gag mutations associated with increased replication capacity.

FIG. 23 presents the distribution of replication capacities observed from set of viruses isolated from treatment-naïve patients for viruses with varying length insertions following the PTAP motif.

FIG. 24 presents a representation of mutations in gag that are associated with increased or decreased replication capacity.

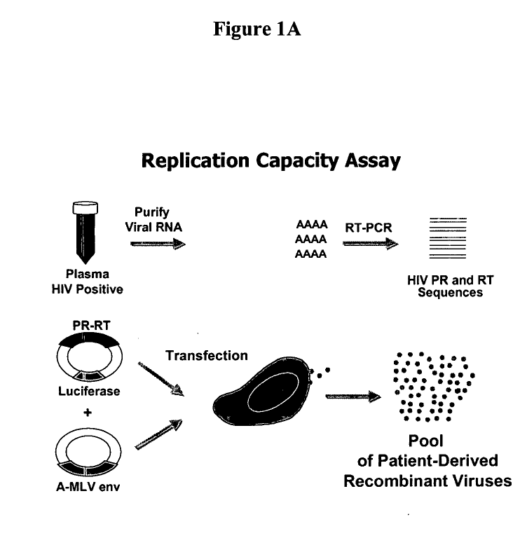

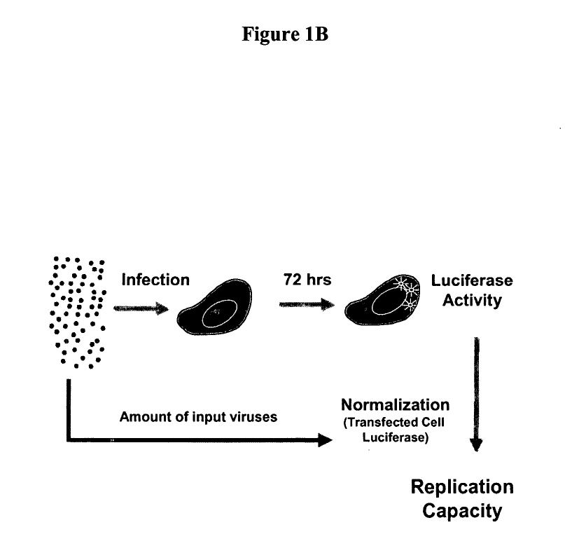

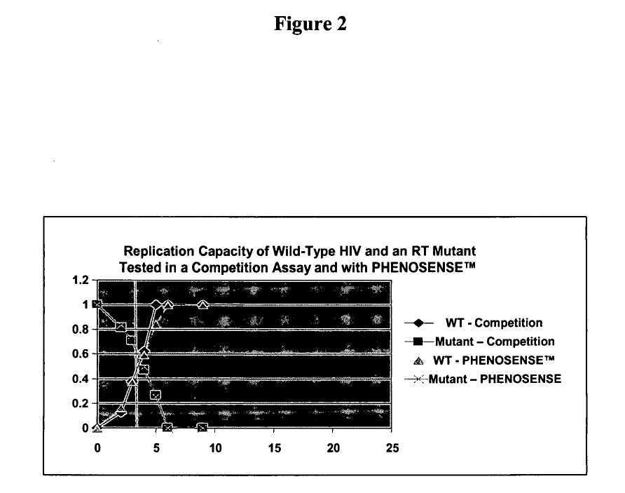

### 5. DETAILED DESCRIPTION OF THE INVENTION

The present invention provides methods for identifying targets for antiviral therapy. In the methods, targets for antiviral therapy can be identified by determining the location of mutations in the viral genome that affect replication capacity. The change in replication capacity indicates that the genetic loci in which the mutations occur are important for essential viral functions, such as replication and/or infectivity. By finely mapping the mutations, specific regions of these genes or their encoded gene products can be identified as attractive targets for antiviral therapy.

5.1. Abbreviations

“NRTI” is an abbreviation for nucleoside reverse transcriptase inhibitor.

“NNRTI” is an abbreviation for non nucleoside reverse transcriptase inhibitor.

“PI” is an abbreviation for protease inhibitor.

“PR” is an abbreviation for protease.

“RT” is an abbreviation for reverse transcriptase.

“PCR” is an abbreviation for “polymerase chain reaction.”

“HBV” is an abbreviation for hepatitis B virus.

“HCV” is an abbreviation for hepatitis C virus.

“HIV” is an abbreviation for human immunodeficiency virus.

The amino acid notations used herein for the twenty genetically encoded L-amino acids are conventional and are as follows:

| | One-Letter | Three Letter |
|---|---|---|
| Amino Acid | Abbreviation | Abbreviation |
| Alanine | A | Ala |
| Arginine | R | Arg |
| Asparagine | N | Asn |
| Aspartic acid | D | Asp |
| Cysteine | C | Cys |
| Glutamine | Q | Gln |
| Glutamic acid | E | Glu |
| Glycine | G | Gly |
| Histidine | H | His |
| Isoleucine | I | Ile |
| Leucine | L | Leu |
| Lysine | K | Lys |
| Methionine | M | Met |
| Phenylalanine | F | Phe |
| Proline | P | Pro |
| Serine | S | Ser |
| Threonine | T | Thr |
| Tryptophan | W | Trp |
| Tyrosine | Y | Tyr |
| Valine | V | Val |

Unless noted otherwise, when polypeptide sequences are presented as a series of one-letter and/or three-letter abbreviations, the sequences are presented in the N->C direction, in accordance with common practice.

Individual amino acids in a sequence are represented herein as AN, wherein A is the standard one letter symbol for the amino acid in the sequence, and N is the position in the sequence. Mutations are represented herein as A₁NA₂, wherein A₁ is the standard one letter symbol for the amino acid in the reference protein sequence, A₂ is the standard one letter symbol for the amino acid in the mutated protein sequence, and N is the position in the amino acid sequence. For example, a G25M mutation represents a change from glycine to methionine at amino acid position 25. Mutations may also be represented herein as NA₂, wherein N is the position in the amino acid sequence and A₂ is the standard one letter symbol for the amino acid in the mutated protein sequence (e.g., 25M, for a change from the wild-type amino acid to methionine at amino acid position 25). Additionally, mutations may also be represented herein as A₁NX, wherein A₁ is the standard one letter symbol for the amino acid in the reference protein sequence, N is the position in the amino acid sequence, and X indicates that the mutated amino acid can be any amino acid (e.g., G25X represents a change from glycine to any amino acid at amino acid position 25). This notation is typically used when the amino acid in the mutated protein sequence is either not known or, if the amino acid in the mutated protein sequence could be any amino acid, except that found in the reference protein sequence, or if the amino acid sequence that is detected is a mixture of two or more different amino acids at the tested position. Further, the notation N- or AN- (e.g., 25- or G25-), where N is the position of the amino acid, indicates that the amino acid at this position is deleted. The amino acid positions are numbered based on the full-length sequence of the protein from which the region encompassing the mutation is derived. Representations of nucleotides and point mutations in DNA sequences are analogous.

The abbreviations used throughout the specification to refer to nucleic acids comprising specific nucleobase sequences are the conventional one-letter abbreviations. Thus, when included in a nucleic acid, the naturally occurring encoding nucleobases are abbreviated as follows: adenine (A), guanine (G), cytosine (C), thymine (T) and uracil (U). Unless specified otherwise, single-stranded nucleic acid sequences that are represented as a series of one-letter abbreviations, and the top strand of double-stranded sequences, are presented in the 5′->3′ direction.

5.2. Definitions

As used herein, the following terms shall have the following meanings:

A “phenotypic assay” is a test that measures a phenotype of a particular virus, such as, for example, HIV, or a population of viruses, such as, for example, the population of HIV infecting a subject. The phenotypes that can be measured include, but are not limited to, the sensitivity of a virus, or of a population of viruses, to a specific anti-viral agent or that measures the replication capacity of a virus.

A “genotypic assay” is an assay that determines a genotype of an organism, a part of an organism, a population of organisms, a gene, a part of a gene, or a population of genes. Typically, a genotypic assay involves determination of the nucleic acid sequence of the relevant gene or genes. Such assays are frequently performed in HIV to establish, for example, whether certain mutations are associated with drug resistance or altered replication capacity are present.

As used herein, “genotypic data” are data about the genotype of, for example, a virus. Examples of genotypic data include, but are not limited to, the nucleotide or amino acid sequence of a virus, a population of viruses, a part of a virus, a viral gene, a part of a viral gene, or the identity of one or more nucleotides or amino acid residues in a viral nucleic acid or protein.

A virus has an “increased likelihood of having altered replication capacity” if the virus has a property, for example, a mutation, that is correlated with an altered replication capacity. A property of a virus is correlated with an altered replication capacity if a population of viruses having the property has, on average, an altered replication capacity relative to that of an otherwise similar population of viruses lacking the property. Thus, the correlation between the presence of the property and altered replication capacity need not be absolute, nor is there a requirement that the property is necessary (i.e., that the property plays a causal role in impairing replication capacity) or sufficient (i.e., that the presence of the property alone is sufficient) for impairing replication capacity.

The terms “replication capacity,” “replication fitness,” and “viral fitness” are used interchangeably and refer to a virus's ability to perform all viral functions necessary to mount a successful infection. Such viral functions include, but are not limited to, entry into the host cell, replication of the viral genome, processing of a viral polyprotein, regulation of viral gene expression, and viral budding to form new viral particles.

The terms “target” and “potential target,” as used herein, refer to a viral molecule, such as, for example, a viral protein, nucleic acid, or lipid, or a portion of a viral molecule such as, for example, a peptide motif or a nucleic acid motif, or combinations of peptide motifs or combinations of peptide motifs, that are identified as affecting replication capacity according to the methods of the invention. The target can encompass a portion of a single molecule. It can also be a combination of viral molecules. The target can also be a combination of one or more viral molecules and one or more molecules from the host cell. Specific examples are provided in the examples, below.

The term “% sequence identity” is used interchangeably herein with the term “% identity” and refers to the level of amino acid sequence identity between two or more peptide sequences or the level of nucleotide sequence identity between two or more nucleotide sequences, when aligned using a sequence alignment program. For example, as used herein, 80% identity means the same thing as 80% sequence identity determined by a defined algorithm, and means that a given sequence is at least 80% identical to another length of another sequence. Exemplary levels of sequence identity include, but are not limited to, 60, 70, 80, 85, 90, 95, 98% or more sequence identity to a given sequence.

The term “% sequence homology” is used interchangeably herein with the term “% homology” and refers to the level of amino acid sequence homology between two or more peptide sequences or the level of nucleotide sequence homology between two or more nucleotide sequences, when aligned using a sequence alignment program. For example, as used herein, 80% homology means the same thing as 80% sequence homology determined by a defined algorithm, and accordingly a homologue of a given sequence has greater than 80% sequence homology over a length of the given sequence. Exemplary levels of sequence homology include, but are not limited to, 60, 70, 80, 85, 90, 95, 98% or more sequence homology to a given sequence.

Exemplary computer programs which can be used to determine identity between two sequences include, but are not limited to, the suite of BLAST programs, e.g., BLASTN, BLASTX, and TBLASTX, BLASTP and TBLASTN, publicly available on the Internet at the NCBI website. See also Altschul et al., 1990, J Mol. Biol. 215:403-10 (with special reference to the published default setting, i.e., parameters w=4, t=17) and Altschul et al., 1997, Nucleic Acids Res., 25:3389-3402. Sequence searches are typically carried out using the BLASTP program when evaluating a given amino acid sequence relative to amino acid sequences in the GenBank Protein Sequences and other public databases. The BLASTX program is preferred for searching nucleic acid sequences that have been translated in all reading frames against amino acid sequences in the GenBank Protein Sequences and other public databases. Both BLASTP and BLASTX are run using default parameters of an open gap penalty of 11.0, and an extended gap penalty of 1.0, and utilize the BLOSUM-62 matrix. See id.

A preferred alignment of selected sequences in order to determine “% identity” between two or more sequences, is performed using for example, the CLUSTAL-W program in MacVector version 6.5, operated with default parameters, including an open gap penalty of 10.0, an extended gap penalty of 0.1, and a BLOSUM 30 similarity matrix.

“Polar Amino Acid” refers to a hydrophilic amino acid having a side chain that is uncharged at physiological pH, but which has at least one bond in which the pair of electrons shared in common by two atoms is held more closely by one of the atoms. Genetically encoded polar amino acids include Asn (N), Gln (Q) Ser (S) and Thr (T).

“Nonpolar Amino Acid” refers to a hydrophobic amino acid having a side chain that is uncharged at physiological pH and which has bonds in which the pair of electrons shared in common by two atoms is generally held equally by each of the two atoms (i.e., the side chain is not polar). Genetically encoded nonpolar amino acids include Ala (A), Gly (G), Ile (I), Leu (L), Met (M) and Val (V).

“Hydrophilic Amino Acid” refers to an amino acid exhibiting a hydrophobicity of less than zero according to the normalized consensus hydrophobicity scale of Eisenberg et al., 1984, J Mol. Biol. 179:125-142. Genetically encoded hydrophilic amino acids include Arg (R), Asn (N), Asp (D), Glu (E), Gln (Q), His (H), Lys (K), Ser (S) and Thr (T).

“Hydrophobic Amino Acid” refers to an amino acid exhibiting a hydrophobicity of greater than zero according to the normalized consensus hydrophobicity scale of Eisenberg et al., 1984, J Mol. Biol. 179:125-142. Genetically encoded hydrophobic amino acids include Ala (A), Gly (G), Ile (I), Leu (L), Met (M), Phe (F), Pro (P), Trp (W), Tyr (Y) and Val (V).

“Acidic Amino Acid” refers to a hydrophilic amino acid having a side chain pK value of less than 7. Acidic amino acids typically have negatively charged side chains at physiological pH due to loss of a hydrogen ion. Genetically encoded acidic amino acids include Asp (D) and Glu (E).

“Basic Amino Acid” refers to a hydrophilic amino acid having a side chain pK value of greater than 7. Basic amino acids typically have positively charged side chains at physiological pH due to association with a hydrogen ion. Genetically encoded basic amino acids include Arg (R), His (H) and Lys (K).

A “mutation” is a change in an amino acid sequence or in a corresponding nucleic acid sequence relative to a reference nucleic acid or polypeptide. For embodiments of the invention comprising HIV protease or reverse transcriptase, the reference nucleic acid encoding protease or reverse transcriptase is the protease or reverse transcriptase coding sequence, respectively, present in NL4-3 HIV (GenBank Accession No. AF324493). Likewise, the reference protease or reverse transcriptase polypeptide is that encoded by the NL4-3 HIV sequence. Although the amino acid sequence of a peptide can be determined directly by, for example, Edman degradation or mass spectroscopy, more typically, the amino sequence of a peptide is inferred from the nucleotide sequence of a nucleic acid that encodes the peptide. Any method for determining the sequence of a nucleic acid known in the art can be used, for example, Maxam-Gilbert sequencing (Maxam et al., 1980, Methods in Enzymology 65:499), dideoxy sequencing (Sanger et al., 1977, Proc. Natl. Acad. Sci. USA 74:5463) or hybridization-based approaches (see e.g., Sambrook et al., 2001, Molecular Cloning: A Laboratory Manual, Cold Spring Harbor Laboratory, 3^{rd} ed., NY; and Ausubel et al., 1989, Current Protocols in Molecular Biology, Greene Publishing Associates and Wiley Interscience, NY).

A “mutant” is a virus, gene or protein having a sequence that has one or more changes relative to a reference virus, gene or protein.

The terms “peptide,” “polypeptide” and “protein” are used interchangeably throughout.

The term “wild-type” refers to a viral genotype that does not comprise a mutation known to be associated with drug resistance.

The terms “polynucleotide,” “oligonucleotide” and “nucleic acid” are used interchangeably throughout.

5.3. Methods of Identifying Targets for Antiviral Therapy

In certain aspects, the present invention provides methods that rely, in part, on identifying mutations associated with altered replication capacity in a virus or a derivative of the virus. Viral mutations, whether associated with resistance to an antiviral drug or otherwise, frequently affect the replication capacity of the virus. See, e.g., Bates et al., 2003, Cur. Opin. Infect. Dis. 16:11-18, which is hereby incorporated by reference in its entirety. Without intending to be bound to any particular theory or mechanism of action, it is believed that these changes in replication capacity associated with mutations reflect changes in the viral genome and encoded gene products that modify the virus's ability to productively enter and reproduce within a cell.

The ability to mount a productive viral infection depends on specific interactions among viral molecules and between such viral molecules and host cell molecules. For example, HIV budding requires interactions between the p6 gag protein and several proteins of the host cell, including Tsg101 and AIP1. Mutations in gag that change the local structure of p6 can either disrupt or potentiate the interaction with these host cell proteins, depending on the nature of the particular mutation. Fine mapping of these mutations can identify the specific residues of p6 that mediate this interaction.

Furthermore, the altered interaction among viral molecules or between viral and host molecules is reflected in changed replication capacity. For example, several gag mutations that map to the specific portions of the p6 gag protein that interact with AIP1 correlate with reduced replication capacity. Conversely, certain insertion mutations in gag that duplicate the p6 gag protein motif that is bound by Tsg101 correlate with increased replication capacity. Thus, by identifying and mapping mutations associated with altered replication capacity, the portions of viral proteins that mediate essential interactions between viral and/or host molecules can be identified.

Such regions of viral proteins present attractive targets for antiviral therapy. After identifying these interactions, modeling algorithms can be used to design antiviral compounds to modulate the interaction. Further, the same phenotypic or genotypic assays that are used to identify the targets for antiviral therapy can be used to assess the effectiveness of the compounds. Any assay that can be used identify compounds that modulate or bind the target that is known to one of skill in the art can also be used to identify such compounds. Alternatively, the phenotypic assays could be used to screen compound libraries to identify compounds that disrupt the essential interactions.

The methods of the invention present several advantages over previous methods for identifying drug targets for antiviral therapy. Principal among such advantages is that they can identify previously unknown interactions among viral molecules or between viral molecules and host cell molecules. Antiviral drugs targeting these novel interactions would provide new classes of antiviral drugs, giving new options for single compound and cocktail antiviral therapies.

Therefore, in certain embodiments, the invention provides a method for identifying a target for antiviral therapy that comprises determining the replication capacity of a statistically significant number of individual viruses, the genotypes of a gene of the statistically significant number of viruses, and a correlation between the replication capacities and the genotypes of the gene, thereby identifying a target for antiviral therapy.

In certain embodiments, the target for antiviral therapy that is identified is a potential target for antiviral therapy that is to be evaluated further. Such further evaluation can comprise, but is not limited to, site-directed mutagenesis, cross-linking studies, derivatization with interfering groups, protection assays, antibody-target interactions, and the like. By using such well-known techniques, the skilled artisan can further evaluate the utility of a target identified using the methods of the invention as a target for antiviral treatment.

In certain embodiments, the replication capacity of the viruses is determined using a phenotypic assay. In certain embodiments, the individual viruses are retroviruses. In further embodiments, the retroviruses are Human Immunodeficiency Viruses (HIV). In other embodiments, the viruses are Hepatitis C viruses (HCV). In yet other embodiments, the viruses are Hepatitis B viruses (HBV). In a preferred embodiment, the retroviruses are HIV.

In certain embodiments, the genotypes that are determined comprise the genotypes of an essential gene of the viruses. In other embodiments, the genotypes that are determined comprise the genotypes of a nonessential gene of the viruses. In yet other embodiments, the genotypes that are determined comprise the genotypes of two or more genes of the viruses.

In certain embodiments, the genotypes that are determined comprise genotypes of an HIV gene that is selected from the group consisting of gag, pol, env, tat, rev, nef, vif, vpr, and vpu, or a combination thereof. In further embodiments, the genotypes that are determined comprise genotypes of gag. In still further embodiments, the genotypes that are determined comprise a genotype of an allele of gag that comprises a mutation, insertion, or deletion.

In certain embodiments, the allele of gag comprises a nucleic acid that encodes a mutation at codo n 418, 427, 429, 437, 439, 442, 454, 465, 466, 470, 473, 478, 482, 483, 484, or 486 of gag, or a combination thereof. In certain embodiments, the mutation is selected from the group consisting of K418R, T427P, I437L, P439S, K442G, E454V, F465Y, T470V, T470Y, S473F, P478L, and L486S, or a combination thereof.

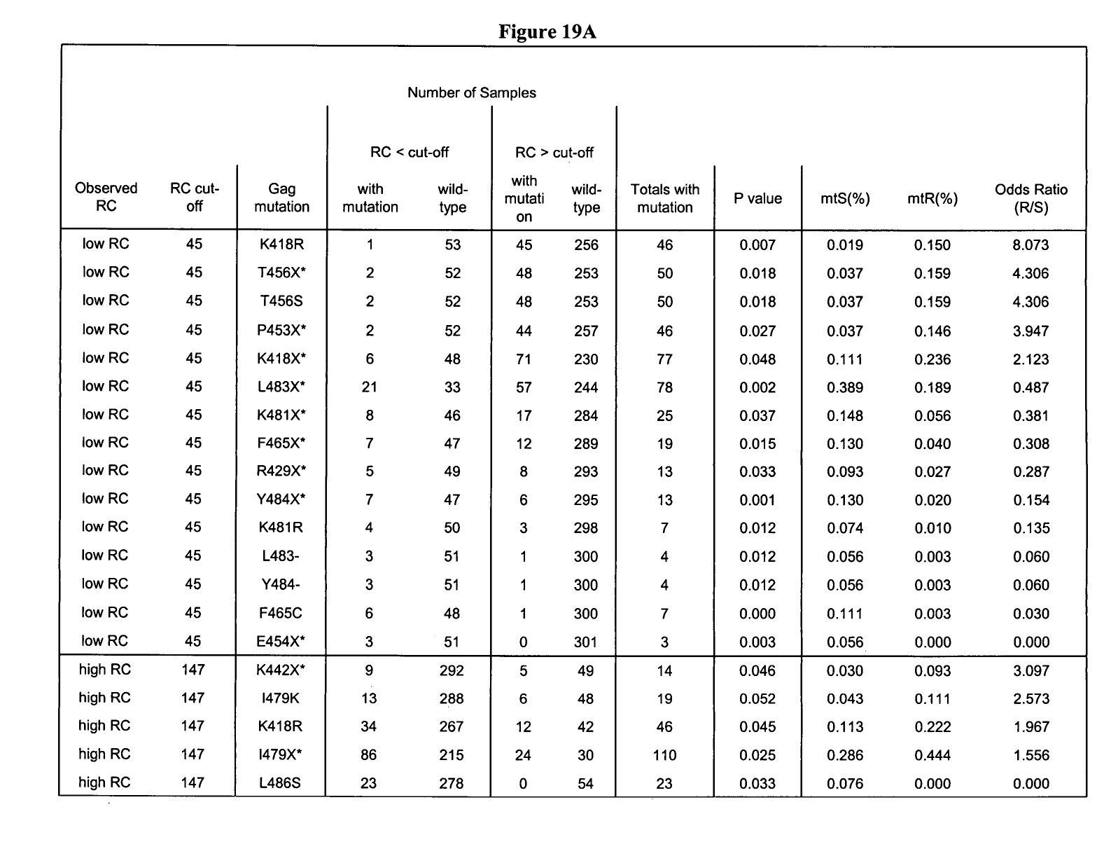

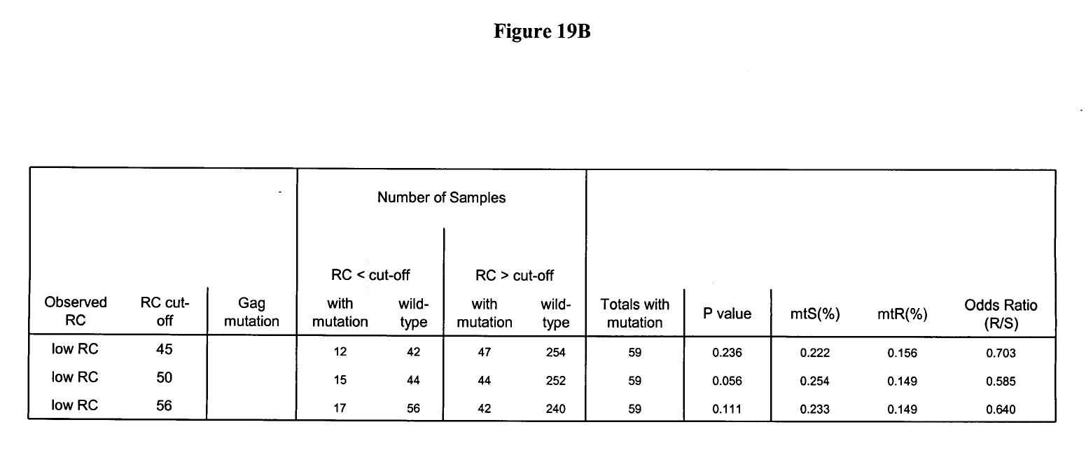

In other embodiments, the allele of gag comprises a nucleic acid that encodes a mutation at codon 418, 439, 454, 473, 478, 481, or 484 of gag, or a combination thereof. In certain embodiments, the mutation is selected from the group consisting of K418R, P439S, E454V, S473F, P478L, and K481E, or a combination thereof. In certain embodiments, the allele of gag comprises a nucleic acid that encodes a mutation in a codon identified in the table of FIG. 19. In certain embodiments, the allele of gag comprises a nucleic acid that encodes a mutation identified in the table of FIG. 19.

In certain embodiments, the allele of gag comprises a nucleic acid that encodes an insertion between codons 460 and 461 of gag or between codons 452 and 453 of gag, or a combination thereof. In further embodiments, the insertion between codons 460 and 461 comprises an insertion of between one and twelve amino acids.

In yet further embodiments, the insertion between codons 460 and 461 of gag comprises an amino acid sequence that has a formula that is X₁-X₂-X₃-X₄-X₅-X₆-X₇-X₈-X₉-X₁₀-X₁₁-X₁₂, wherein:

X₁ is selected from the group consisting of P, R, E, Q, and T;

X₂ is absent or selected from the group consisting of P, R, A, S, and T;

X₃ is absent or selected from the group consisting of E, A, F, P, T, and R;

X₄ is absent or selected from the group consisting of P, R, A, and E;

X₅ is absent or selected from the group consisting of P, A, E, and T;

X₆ is absent or selected from the group consisting of A, E, P, Q, T, and V;

X₇ is absent or selected from the group consisting of P, T, and A;

X₈ is absent or selected from the group consisting of P, T, and A;

X₉ is absent or selected from the group consisting of P and A;

X₁₀ is absent or selected from the group consisting of P and E;

X₁₁ is absent or selected from the group consisting of P and E;

X₁₂ is absent or R.

In still further embodiments, the insertion between codons 460 and 461 of gag comprises an amino acid sequence that is selected from the group of E, PE, PPE, PPA, TAPPA, PTAPPA, PTAPPE, EPTAPP, PTAPPQ, PSAPPE, PTAPPV, and RPEPTAPPA.

In other embodiments, the insertion between codons 452 and 453 of gag comprises an insertion of between two and ten amino acids.

In further embodiments, the insertion between codons 452 and 453 of gag comprises an amino acid sequence that has a formula that is X₁-X₂-X₃-X₄-X₅-X₆-X₇-X₈-X₉-X₁₀, wherein:

X₁ is selected from the group consisting of P, S, and T;

X₂ is selected from the group consisting of R, D, E, Q, and S;

X₃ is absent or selected from the group consisting of P, S, Q, and N;

X₄ is absent or selected from the group consisting of R, Q, T, and S;

X₅ is absent or selected from the group consisting of P, A, R, and S;

X₆ is absent or selected from the group consisting of R and P;

X₇ is absent or selected from the group consisting of R, P, L, S, and Q

X₈ is absent or selected from the group consisting of Q, R, and S;

X₉ is absent or selected from the group consisting of S and R; and

X₁₀is absent or R.

In yet further embodiments, the insertion between codons 452 and 453 of gag comprises an amino acid sequence that is selected from the group consisting of SR, SS, PEP, PESR, PEPR, PQSR, TENR, PDQSR, PEPSR, PEQSR, PEPSAR, PEPQSR, PQPTAP, PEPTAR, PEPTAPR, PEPTAPSR and PEPTAPLQSR.

In other embodiments, the allele of gag comprises an insertion between codons 458 and 459 of gag. In certain embodiments, the insertion between codons 458 and 459 of gag comprises an insertion of between three and fourteen amino acids. In further embodiments, the insertion between codons 458 and 459 of gag comprises an amino acid sequence that has a formula that is X₁-X₂-X₃-X₄-X₅-X₆, wherein:

X₁ is absent or selected from the group consisting of P and T;

X₂ is absent or E;

X₃ is absent or P;

X₄ is selected from the group consisting of P, S, and T;

X₅ is A; and

X₆ is P.

In certain embodiments, the insertion between codons 458 and 459 of gag comprises an amino acid sequence that is selected from the group of PEPSAP, TEPTAP, PEPTAP, EPTAP, PXAP, PAP, SAP, and TAP.

In certain embodiments, the genotypes that are determined comprise genotypes of pol. In further embodiments, the genotypes that are determined comprise a genotype of an allele of pol that comprises a mutation, insertion, or deletion.

In certain embodiments, the allele of pol comprises a mutation in the region of pol that encodes protease. In further embodiments, the mutation is selected from the group consisting of mutations at codons 10, 14, 15, 20, 36, 37, 39, 61, 63, 64, 71, 72, 77, and 93 of protease, or a combination thereof. In still further embodiments, the mutation is selected from the group consisting of I15V, K20M, M36L, N37D, P39Q, P39S, Q61N, A71T, and V77I, or a combination thereof.

In other embodiments, the allele of pol comprises a mutation in the region of pol that encodes reverse transcriptase. In further embodiments, the mutation is selected from the group consisting of mutations at codons 39, 121, 135, 138, 196, 203, 204, 207, 210, 211, 245, 248, 275, 276, and 286, or a combination thereof. In still further embodiments, the mutation is selected from the group consisting of D121Y, I135V, E138A, G196E, E203D, E204D, E204K, Q207E, R211Q, V245E, E248D, K275Q, V276T, and T286P, or a combination thereof.

In other embodiments, the genotypes that are determined comprise genotypes of a 5′ or 3′ untranslated region.

In certain embodiments, the at least one target that is identified comprises a nucleic acid that encodes a portion of gag, pol, env, tat, rev, nef, vif, vpr, and vpu. In other embodiments, the at least one target that is identified is a nucleic acid that comprises a portion of a 5′ or 3′ untranslated region.

In certain embodiments, the at least one target that is identified comprises a portion of a viral protein that interacts with a host cell protein. In other embodiments, the at least one target that is identified comprises a portion of a first viral protein that interacts with a second viral protein. In certain of these embodiments, the first viral protein is the same protein as the second viral protein.

In certain embodiments, the at least one target that is identified comprises a primary structure motif. In other embodiments, the at least one target that is identified comprises a secondary structure motif. In yet other embodiments, the at least one target that is identified comprises a tertiary structure motif. In still other embodiments, the at least one target that is identified comprises a quaternary structure motif.

In certain embodiments, the at least one target that is identified comprises a portion of a protein that is selected from the group consisting of p1 gag protein, p2 gag protein, p6* pol protein, p6 gag protein, p7 nucleocapsid protein, p17 matrix protein, p24 capsid protein, p55 gag protein, p10 protease, p66 reverse transcriptase/RNAse H, p51 reverse transcriptase, p32 integrase, gp120 envelope glycoprotein, gp41 glycoprotein, p23 vif protein, p15 vpr protein, p14 tat protein, p19 rev protein, p27 nef protein, p16 vpu protein, and p12-16 vpx protein, or a combination thereof.

In further embodiments, the one target that is identified comprises a portion of gag. In yet further embodiments, the portion of gag comprises a PTAP motif. In still further embodiments, the PTAP motif is at positions 455-458 of gag. In other embodiments, the portion of gag comprises a LYP or LRSL motif. In still other embodiments, the portion of gag comprises an amino acid that is selected from the group consisting of residues 418, 427, 429, 437, 439, 442, 454, 465, 466, 470, 473, 478, 482, 483, 484, or 486 of gag, or a combination thereof. In further embodiments, the portion of gag comprises residue 484 of gag. In certain embodiments, the portion of gag that is identified does not comprise a motif that binds Tsg101. In other embodiments, the portion of gag that is identified does not comprise a motif that binds AIP1. In other embodiments, the portion of gag comprises a portion of gag that is selected from the group consisting of residues 418-429, residues 427-437, residues 439-442, residues 439-454, residues 454-466, residues 454-470, residues 465-473, residues 465-478, residues 470-478, residues 470-486, residues 478-486, and residues 482-486, or a combination thereof.

In other embodiments, the at least one target that is identified comprises a portion of protease. In certain embodiments, the portion of protease comprises an amino acid selected from the group consisting of residues 10, 14, 15, 20, 36, 37, 39, 61, 63, 64, 71, 72, 77, and 93 of protease, or a combination thereof. In other embodiments, the portion of protease comprises a portion of protease that is selected from the group consisting of residues 10-15, residues 10-20, residues 14-20, residues 20-39, residues 36-39, residues 10-39, residues 61-77, residues 61-64, residues 61-72, residues 71-77, and residues 71-93, or a combination thereof.

In other embodiments, the at least one target that is identified comprises a portion of reverse transcriptase. In certain embodiments, the portion of reverse transcriptase comprises an amino acid that is selected from the group consisting of residues 39, 121, 135, 138, 196, 203, 204, 207, 210, 211, 245, 248, 275, 276, and 286 of reverse transcriptase, or a combination thereof. In other embodiments, the portion of reverse transcriptase comprises a portion of reverse transcriptase that is selected from the group consisting of residues 121- 138, residues 196-211, residues 245-248, and residues 275-286, or a combination thereof.

In still other embodiments, the viruses whose genotypes and phenotypes are determined and correlated are Hepatitis C viruses. In certain embodiments, the genotypes that are determined comprise genotypes of a region of a Hepatitis C viral genome that are selected from the group consisting of a 5′ untranslated region, a polyprotein-encoding region, and a 3′ untranslated region. In further embodiments, the HCV genotypes that are determined comprise the genotypes of the polyprotein-encoding region. In still further embodiments, the HCV genotypes that are determined comprise the genotypes of a gene that encodes a protein selected from the group consisting of C, E1, E2, p7, NS2, NS3, NS4A, NS4B, NS5A, and NS5B.

In certain embodiments, the at least one target that is identified comprises a nucleic acid that encodes a portion of a Hepatitis C viral polyprotein. In further embodiments, the at least one target that is identified comprises a portion of a Hepatitis C viral protein that is selected from the group consisting of C, E1, E2, p7, NS2, NS3, NS4A, NS4B, NS5A, and NS5B. In other embodiments, the at least one target that is identified is a nucleic acid that comprises a portion of a Hepatitis C viral genome that is selected from the group consisting of a 5′ untranslated region and a 3′ untranslated region.

In other embodiments, the viruses whose genotypes and phenotypes are determined and correlated are hepadnaviruses. In further embodiments, the hepadnaviruses are hepatitis B viruses.

In certain embodiments, the genotypes that are determined comprise genotypes of a region of a hepatitis B viral genome that is selected from the group consisting of a 5′ untranslated region and a 3′ untranslated region. In other embodiments, the HBV genotypes that are determined comprise genotypes of a gene that is selected from the group consisting of pre-S1, pre-S2, S, C, P and X genes.

In certain embodiments, the at least one target that is identified comprises a portion of a nucleic acid that encodes a Hepatitis B protein that is selected from the group consisting of pre-S1, pre-S2, S, C, P and X. In other embodiments, the at least one target that is identified is a nucleic acid that comprises a portion of a Hepatitis B viral genome that is selected from the group consisting of a 5′ untranslated region and a 3′ untranslated region. In still other embodiments, the at least one target that is identified comprises a portion of a Hepatitis B protein that is selected from the group consisting of pre-S1, pre-S2, S, C, P and X.

5.4. Measuring Replication Capacity of a Virus with a Phenotypic Assay

Any method known in the art can be used to determine a viral replication capacity phenotype, without limitation. See e.g., U.S. Pat. Nos. 5,837,464 and 6,242,187, each of which is hereby incorporated by reference in its entirety.

In certain embodiments, the phenotypic analysis is performed using recombinant virus assays (“RVAs”). RVAs use virus stocks generated by homologous recombination between viral vectors and viral gene sequences, amplified from the patient virus. In certain embodiments, the viral vector is a HIV vector and the viral gene sequences are protease and/or reverse transcriptase and/or gag sequences.

In preferred embodiments, the phenotypic analysis of replication capacity is performed using PHENOSENSE™ (ViroLogic Inc., South San Francisco, Calif.). See Petropoulos et al., 2000, Antimicrob. Agents Chemother. 44:920-928; U.S. Pat. Nos. 5,837,464 and 6,242,187. PHENOSENSE™ is a phenotypic assay that achieves the benefits of phenotypic testing and overcomes the drawbacks of previous assays. Because the assay has been automated, PHENOSENSE™ provides high throughput methods under controlled conditions for determining replication capacity of a large number of individual viral isolates.

The result is an assay that can quickly and accurately define both the replication capacity and the susceptibility profile of a patient's HIV (or other virus) isolates to all currently available antiretroviral drugs. PHENOSENSE™ can obtain results with only one round of viral replication, thereby avoiding selection of subpopulations of virus that can occur during preparation of viral stocks required for assays that rely on fully infectious virus. Further, the results are both quantitative, measuring varying degrees of replication capacity, and sensitive, as the test can be performed on blood specimens with a viral load of about 500 copies/mL and can detect minority populations of some drug-resistant virus at concentrations of 10% or less of total viral population. Finally, the replication capacity results are reproducible and can vary by less than about 0.25 logs in about 95% of the assays performed.

PHENOSENSE™ can be used with nucleic acids from amplified viral gene sequences. As discussed in Section 5.4.1, the nucleic acid can be amplified from any sample known by one of skill in the art to contain a viral gene sequence, without limitation. For example, the sample can be a sample from a human or an animal infected with the virus or a sample from a culture of viral cells. In certain embodiments, the viral sample comprises a genetically modified laboratory strain. In other embodiments, the viral sample comprises a wild-type isolate.

A resistance test vector (“RTV”) can then be constructed by incorporating the amplified viral gene sequences into a replication defective viral vector by using any method known in the art of incorporating gene sequences into a vector. In one embodiment, restrictions enzymes and conventional cloning methods are used. See Sambrook et al., 2001, Molecular Cloning: A Laboratory Manual, Cold Spring Harbor Laboratory, 3^{rd} ed., NY; and Ausubel et al., 1989, Current Protocols in Molecular Biology, Greene Publishing Associates and Wiley Interscience, NY. In a preferred embodiment, ApaI and PinAI restriction enzymes are used. Preferably, the replication defective viral vector is the indicator gene viral vector (“IGVV”). In a preferred embodiment, the viral vector contains a means for detecting replication of the RTV. Preferably, the viral vector contains a luciferase expression cassette.

The assay can be performed by first co-transfecting host cells with RTV DNA and a plasmid that expresses the envelope proteins of another retrovirus, for example, amphotropic murine leukemia virus (MLV). Following transfection, viral particles can be harvested from the cell culture and used to infect fresh target cells. The completion of a single round of viral replication in the fresh target cells can be detected by the means for detecting replication contained in the vector. In a preferred embodiment, the completion of a single round of viral replication results in the production of luciferase.

Replication capacity of the virus can be measured by assessing the amount of indicator gene activity observed in the target cells. For example, replication capacity can be measured by determining the amount of luciferase activity in target cell when the indicator gene is luciferase. In such systems, cells infected with viruses with high replication capacity exhibit more luciferase activity, while cells infected with viruses with low replication capacity exhibit less luciferase activity.

More specifically, in certain embodiments, virus can be classified as having low, medium, or high replication capacity. In certain embodiments, a virus with low replication capacity exhibits a replication capacity that is less than about 15%, less than about 20%, less than about 25%, less than about 30%, less than about 35%, less than about 40%, less than about 45%, less than about 50%, less than about 55%, less than about 60%, less than about 65%, less than about 70%, or less than about 75% of the median replication capacity observed in a statistically significant number of individual viral isolates. In a preferred embodiment, a virus with low replication capacity exhibits a replication capacity that is less than about 54% of the median replication capacity observed in a statistically significant number of individual viral isolates.

One of skill in the art can readily recognize how many individual viruses' replication capacities should be evaluated in order for the number of viruses to be statistically significant. For example, the statistical methods presented in the examples, below, can be used to determine whether the viral sample size is large enough for a correlation identified between replication capacity and genotype to be significant.

In certain embodiments, a virus with medium replication capacity exhibits a replication capacity that is between about 75% and about 125%, between about 80% and about 120%, between about 85% and about 115%, between about 90% and about 110%, between about 95% and about 105%, between about 97% and about 102%, between about 94% and 101%, or between about 95% and about 98% of the median replication capacity observed in a statistically significant number of individual viral isolates.

In certain embodiments, a virus with high replication capacity exhibits a replication capacity that is greater than about 125%, greater than about 130%, greater than about 135%, greater than about 140%, greater than about 145%, greater than about 150%, greater than about 155%, greater than about 160%, greater than about 165%, greater than about 170%, or greater than about 175% of the median replication capacity observed in a statistically significant number of individual viral isolates. In a preferred embodiment, a virus with low replication capacity exhibits a replication capacity that is greater than about 180% of the median replication capacity observed in a statistically significant number of individual viral isolates.

In other embodiments, a virus can be classified as having low, medium, or high replication capacity based upon its presence in a given percentile of observed replication capacities for a statistically significant number of viruses. For example, a virus that has a replication capacity that is in the bottom 10% of total replication capacities measured, if a statistically significant number of such capacities are measured, could be considered to have low replication capacity. Similarly, a virus that has a replication capacity that is in the top 90% of a replication capacities measured would be an example of a virus that could be considered to have high replication capacity.

Thus, in certain embodiments, a virus has a low replication capacity if its replication capacity is in about the 1^{st} percentile, about the 2^{nd} percentile, about the 3^{rd} percentile, about the 4^{th} percentile, about the 5^{th} percentile, about the 6^{th} percentile, about the 7^{th} percentile, about the 8^{th} percentile, about the 9^{th} percentile, about the 10^{th} percentile, about the 15^{th} percentile, or about the 20^{th} percentile of replication capacities measured for a statistically significant number of viruses. In a preferred embodiment, a virus has a low replication capacity if its replication capacity is in about the 10^{th} percentile of replication capacities measured of a statistically significant number of viruses.

In certain embodiments, a virus has a high replication capacity if its replication capacity is in about the 80^{th} percentile, about the 85^{th} percentile, about the 90^{th} percentile, about the 91^{st} percentile, about the 92^{nd} percentile, about the ₉₃rd percentile, about the 94^{th} percentile, about the 95^{th} percentile, about the 96^{th} percentile, about the 97^{th} percentile, about the 98^{th} percentile, or about the 99^{th} percentile of replication capacities measured for a statistically significant number of viruses. In a preferred embodiment, a virus has a high replication capacity if its replication capacity is in about the 90^{th} percentile of replication capacities measured of a statistically significant number of viruses.

In preferred embodiments, PHENOSENSE™ is used to evaluate the replication capacity phenotype of HIV-1. In other embodiments, PHENOSENSE™ is used to evaluate the replication capacity phenotype of HIV-2. In certain embodiments, the HIV-1 strain that is evaluated is a wild-type isolate of HIV-1. In other embodiments, the HIV-1 strain that is evaluated is a mutant strain of HIV-1. In certain embodiments, such mutant strains can be isolated from patients. In other embodiments, the mutant strains can be constructed by site-directed mutagenesis or other equivalent techniques known to one of skill in the art.

In one embodiment, viral nucleic acid, for example, HIV-1 RNA is extracted from plasma samples, and a fragment of, or entire viral genes can be amplified by methods such as, but not limited to PCR. See, e.g., Hertogs et al., 1998, Antimicrob Agents Chemother 42(2):269-76. In one example, a 2.2-kb fragment containing the entire HIV-1 PR- and RT-coding sequence is amplified by nested reverse transcription-PCR. The pool of amplified nucleic acid, for example, the PR-RT-coding sequences, is then cotransfected into a host cell such as CD4+ T lymphocytes (MT4) with the pGEMT3deltaPRT plasmid from which most of the PR (codons 10 to 99) and RT (codons 1 to 482) sequences are deleted. Homologous recombination leads to the generation of chimeric viruses containing viral coding sequences, such as the PR- and RT-coding sequences derived from HIV-1 RNA in plasma. The replication capacities of the chimeric viruses can be determined by any cell viability assay known in the art, and compared to replication capacities of a statistically significant number of individual viral isolates to assess whether a virus has. For example, an MT4 cell-3-(4,5-dimethylthiazol-2-yl) -2,5-diphenyltetrazolium bromide-based cell viability assay can be used in an automated system that allows high sample throughput.

In another embodiment, competition assays can be used to assess replication capacity of one viral strain relative to another viral strain. For example, two infectious viral strains can be co-cultivated together in the same culture medium. See, e.g., Lu et al., 2001, JAIDS 27:7-13, which is incorporated by reference in its entirety. By monitoring the course of each viral strain's growth, the fitness of one strain relative to the other can be determined. By measuring many viruses' fitness relative to a single reference virus, an objective measure of each strain's fitness can be determined. These measurements of replication capacity can then be used according to the methods of the invention to identify targets for antiviral therapy.

Other assays for evaluating the phenotypic susceptibility of a virus to anti-viral drugs known to one of skill in the art can be adapted to determine replication capacity. See, e.g., Shi and Mellors, 1997, Antimicrob Agents Chemother. 41(12):2781-85; Gervaix et al., 1997, Proc Natl Acad Sci U.S.A. 94(9):4653-8; Race et al., 1999, AIDS 13:2061-2068, incorporated herein by reference in their entireties.

5.4.1. Detecting the Presence or Absence of Mutations in a Virus

The presence or absence of an altered replication capacity-associated mutation according to the present invention in a virus can be determined by any means known in the art for detecting a mutation. The mutation can be detected in the viral gene that encodes a particular protein, or in the protein itself, i.e., in the amino acid sequence of the protein.

In one embodiment, the mutation is in the viral genome. Such a mutation can be in, for example, a gene encoding a viral protein, in a genetic element such as a cis or trans acting regulatory sequence of a gene encoding a viral protein, an intergenic sequence, or an intron sequence. The mutation can affect any aspect of the structure, function, replication or environment of the virus that changes its susceptibility to an anti-viral treatment and/or its replication capacity. In one embodiment, the mutation is in a gene encoding a viral protein that is the target of an currently available anti-viral treatment. In other embodiments, the mutation is in a gene or other genetic element that is not the target of a currently-available anti-viral treatment.

A mutation within a viral gene can be detected by utilizing any suitable technique known to one of skill in the art without limitation. Viral DNA or RNA can be used as the starting point for such assay techniques, and may be isolated according to standard procedures which are well known to those of skill in the art.

The detection of a mutation in specific nucleic acid sequences, such as in a particular region of a viral gene, can be accomplished by a variety of methods including, but not limited to, restriction-fragment-length-polymorphism detection based on allele-specific restriction-endonuclease cleavage (Kan and Dozy, 1978, Lancet ii:910-912), mismatch-repair detection (Faham and Cox, 1995, Genome Res 5:474-482), binding of MutS protein (Wagner et al., 1995, Nucl Acids Res 23:3944-3948), denaturing-gradient gel electrophoresis (Fisher et al., 1983, Proc. Natl. Acad. Sci. U.S.A. 80:1579-83), single-strand-conformation-polymorphism detection (Orita et al., 1983, Genomics 5:874-879), RNAase cleavage at mismatched base-pairs (Myers et al., 1985, Science 230:1242), chemical (Cotton et al., 1988, Proc. Natl. Acad. Sci. U.S.A. 85:4397-4401) or enzymatic (Youil et al., 1995, Proc. Natl. Acad. Sci. U.S.A. 92:87-91) cleavage of heteroduplex DNA, methods based on oligonucleotide-specific primer extension (Syvanen et al., 1990, Genomics 8:684-692), genetic bit analysis (Nikiforov et al., 1994, Nucl Acids Res 22:4167-4175), oligonucleotide-ligation assay (Landegren et al., 1988, Science 241:1077), oligonucleotide-specific ligation chain reaction (“LCR”) (Barrany, 1991, Proc. Natl. Acad. Sci. U.S.A. 88:189-193), gap-LCR (Abravaya et al., 1995, Nucl Acids Res 23:675-682), radioactive or fluorescent DNA sequencing using standard procedures well known in the art, and peptide nucleic acid (PNA) assays (Orum et al., 1993, Nucl. Acids Res. 21:5332-5356; Thiede et al., 1996, Nucl. Acids Res. 24:983-984).

In addition, viral DNA or RNA may be used in hybridization or amplification assays to detect abnormalities involving gene structure, including point mutations, insertions, deletions and genomic rearrangements. Such assays may include, but are not limited to, Southern analyses (Southern, 1975, J Mol. Biol. 98:503-517), single stranded conformational polymorphism analyses (SSCP) (Orita et al., 1989, Proc. Natl. Acad. Sci. USA 86:2766-2770), and PCR analyses (U.S. Pat. Nos. 4,683,202; 4,683,195; 4,800,159; and 4,965,188; PCR Strategies, 1995 Innis et al. (eds.), Academic Press, Inc.).

Such diagnostic methods for the detection of a gene-specific mutation can involve for example, contacting and incubating the viral nucleic acids with one or more labeled nucleic acid reagents including recombinant DNA molecules, cloned genes or degenerate variants thereof, under conditions favorable for the specific annealing of these reagents to their complementary sequences. Preferably, the lengths of these nucleic acid reagents are at least 15 to 30 nucleotides. After incubation, all non-annealed nucleic acids are removed from the nucleic acid molecule hybrid. The presence of nucleic acids which have hybridized, if any such molecules exist, is then detected. Using such a detection scheme, the nucleic acid from the virus can be immobilized, for example, to a solid support such as a membrane, or a plastic surface such as that on a microtiter plate or polystyrene beads. In this case, after incubation, non-annealed, labeled nucleic acid reagents of the type described above are easily removed. Detection of the remaining, annealed, labeled nucleic acid reagents is accomplished using standard techniques well-known to those in the art. The gene sequences to which the nucleic acid reagents have annealed can be compared to the annealing pattern expected from a normal gene sequence in order to determine whether a gene mutation is present.

These techniques can easily be adapted to provide high-throughput methods for detecting mutations in viral genomes. For example, a gene array from Affymetrix (Affymetrix, Inc., Sunnyvale, Calif.) can be used to rapidly identify genotypes of a large number of individual viruses. Affymetrix gene arrays, and methods of making and using such arrays, are described in, for example, U.S. Pat. Nos. 6,551,784, 6,548,257, 6,505,125, 6,489,114, 6,451,536, 6,410,229, 6,391,550, 6,379,895, 6,355,432, 6,342,355, 6,333,155, 6,308,170, 6,291,183, 6,287,850, 6,261,776, 6,225,625, 6,197,506, 6,168,948, 6,156,501, 6,141,096, 6,040,138, 6,022,963, 5,919,523, 5,837,832, 5,744,305, 5,834,758, and 5,631,734, each of which is hereby incorporated by reference in its entirety.

In addition, Ausubel et al., eds., Current Protocols in Molecular Biology, 2002, Vol. 4, Unit 25B, Ch. 22, which is hereby incorporated by reference in its entirety, provides further guidance on construction and use of a gene array for determining the genotypes of a large number of viral isolates. Finally, U.S. Pat. Nos. 6,670,124; 6,617,112; 6,309,823; 6,284,465; and 5,723,320, each of which is incorporated by reference in its entirety, describe related array technologies that can readily be adapted for rapid identification of a large number of viral genotypes by one of skill in the art.

Alternative diagnostic methods for the detection of gene specific nucleic acid molecules may involve their amplification, e.g., by PCR (U.S. Pat. Nos. 4,683,202; 4,683,195; 4,800,159; and 4,965,188; PCR Strategies, 1995 Innis et al. (eds.), Academic Press, Inc.), followed by the detection of the amplified molecules using techniques well known to those of skill in the art. The resulting amplified sequences can be compared to those which would be expected if the nucleic acid being amplified contained only normal copies of the respective gene in order to determine whether a gene mutation exists.

Additionally, the nucleic acid can be sequenced by any sequencing method known in the art. For example, the viral DNA can be sequenced by the dideoxy method of Sanger et al., 1977, Proc. Natl. Acad. Sci. USA 74:5463, as further described by Messing et al., 1981, Nuc. Acids Res. 9:309, or by the method of Maxam et al., 1980, Methods in Enzymology 65:499. See also the techniques described in Sambrook et al., 2001, Molecular Cloning: A Laboratory Manual, Cold Spring Harbor Laboratory, 3^{rd} ed., NY; and Ausubel et al., 1989, Current Protocols in Molecular Biology, Greene Publishing Associates and Wiley Interscience, NY.

Antibodies directed against the viral gene products, i.e., viral proteins or viral peptide fragments can also be used to detect mutations in the viral proteins. Alternatively, the viral protein or peptide fragments of interest can be sequenced by any sequencing method known in the art in order to yield the amino acid sequence of the protein of interest. An example of such a method is the Edman degradation method which can be used to sequence small proteins or polypeptides. Larger proteins can be initially cleaved by chemical or enzymatic reagents known in the art, for example, cyanogen bromide, hydroxylamine, trypsin or chymotrypsin, and then sequenced by the Edman degradation method.

5.4.2. Correlating Mutations with their Effects on Replication Capacity

Any method known in the art can be used to determine whether a mutation is correlated with an altered replication capacity. Such methods can be applied to variously constructed sets of mutations and/or replication capacities. In certain embodiments, the methods are applied to viruses that have replication capacities that appear in particular percentiles of all replication capacities observed for a statistically significant number of viruses. For example, in certain embodiments, the methods can be applied to the viruses that appear in the bottom 10% of observed replication capacities. In other embodiments, the methods can be applied to viruses that appear in the top 10% of observed replication capacities. In still other embodiments, the methods can be applied to the viruses that appear in either the top or the bottom 10% of observed replication capacities.

In one embodiment, univariate analysis is used to identify mutations correlated with altered replication capacity. Univariate analysis yields P values that indicate the statistical significance of the correlation. In such embodiments, the smaller the P value, the more significant the measurement. Preferably the P values will be less than 0.05. More preferably, P values will be less than 0.01. Even more preferably, the P value will be less than 0.005. P values can be calculated by any means known to one of skill in the art. In one embodiment, P values are calculated using Fisher's Exact Test. In another embodiment, P values can be calculated with Student's t-test. See, e.g., David Freedman, Robert Pisani & Roger Purves, 1980, STATISTICS, W. W. Norton, New York. In certain embodiments, P values can be calculated with both Fisher's Exact Test and Student's t-test. In such embodiments, P values calculated with both tests are preferably less than 0.05. However, a correlation with a P value that is less than 0.10 in one test but less than 0.05 in another test can still be considered to be a marginally significant correlation. Such mutations are suitable for further analysis with, for example, multivariate analysis. Alternatively, further univariate analysis can be performed on a larger sample set to confirm the significance of the correlation.

Further, an odds ratio can be calculated to determine whether a mutation associated with altered replication capacity correlates with high or low replication capacity. In certain embodiments, an odds ratio that is greater than one indicates that the mutation correlates with high replication capacity. In certain embodiments, an odds ratio that is less than one indicates that the mutation correlates with low replication capacity.

In yet another embodiment, multivariate analysis can be used to determine whether a mutation correlates with altered replication capacity. Any multivariate analysis known by one of skill in the art to be useful in calculating such a correlation can be used, without limitation. In certain embodiments, a statistically significant number of virus's replication capacities can be determined. These replication capacities can then be divided into groups that correspond to percentiles of the set of replication capacities observed. For example, and not by way of limitation, the replication capacities can be divided up into 21 groups. Each group corresponds to about 4.75% of the total replication capacities observed.

After assigning each virus's replication capacity to the appropriate group, the genotype of that virus can be assigned to that group. For example, and not by way of limitation, one virus that has a replication capacity in the lowest 4.75% of replication capacities observed is a virus that comprises a mutation in codon 478 of gag. More particularly, this example virus comprises the mutation P478L. Thus, this instance of this mutation is assigned to the lowest 4.75% of replication capacities observed. Any other mutation(s) detected in this example virus would also be assigned to this percentile. By performing this method for all viral isolates, the number of instances of a particular mutation in a given percentile of replication capacity can be observed. This allows the skilled practitioner to identify mutations that correlate with altered replication capacity.

Finally, in yet another embodiment, regression analysis can be performed to identify mutations that best predict altered replication capacity. In such embodiments, regression analysis is performed on a statistically significant number of viral isolates for which genotypes and replication capacities have been determined. The analysis then identifies which mutations appear to best predict, e.g., most strongly correlate with, altered replication capacity. Such analysis can then be used to construct rules for predicting replication capacity based upon knowledge of the genotype of a particular virus, described below.

5.5. Methods for Predicting Replication Capacity Based on Viral Genotype

In another aspect, the present invention provides methods for predicting a virus's replication capacity based upon the presence of particular mutations in the viral genome. In certain embodiments, the methods are based, in part, on the results of regression analysis of mutations correlated with altered replication capacity as described above. In other embodiments, the methods are based, in part, on the results of univariate analysis of mutations correlated with altered replication capacity. In yet other embodiments, the methods are based, in part, on the results of multivariate analysis of mutations correlated with altered replication capacity.

Thus, in certain embodiments, the invention provides a method for determining that an HIV has altered replication capacity that comprises detecting a mutation in a codon of gag that is selected from the group consisting of codons 418, 427, 429, 437, 439, 442, 454, 465, 466, 470, 473, 478, 482, 483, 484, and 486, or any combination thereof. In certain embodiments, the mutation is in codon 418 of gag. In certain embodiments, the mutation is in codon 427 of gag. In certain embodiments, the mutation is in codon 429 of gag. In certain embodiments, the mutation is in codon 437 of gag. In certain embodiments, the mutation is in codon 439 of gag. In certain embodiments, the mutation is in codon 442 of gag. In certain embodiments, the mutation is in codon 454 of gag. In certain embodiments, the mutation is in codon 465 of gag. In certain embodiments, the mutation is in codon 466 of gag. In certain embodiments, the mutation is in codon 470 of gag. In certain embodiments, the mutation is in codon 473 of gag. In certain embodiments, the mutation is in codon 473 of gag. In certain embodiments, the mutation is in codon 478 of gag. In certain embodiments, the mutation is in codon 482 of gag. In certain embodiments, the mutation is in codon 483 of gag. In certain embodiments, the mutation is in codon 484 of gag. In certain embodiments, the mutation is in codon 486 of gag.

In certain embodiments, the mutation can be selected from the group consisting of K418R, T427P, I437L, P439S, K442G, E454V, F465Y, T470V, T470Y, S473F, P478L, and L486S, or any combination thereof. In certain embodiments, the mutation is K418R In certain embodiments, the mutation is T427P. In certain embodiments, the mutation is I437L. In certain embodiments, the mutation is P439S. In certain embodiments, the mutation is K442G. In certain embodiments, the mutation is K442G. In certain embodiments, the mutation is E454V. In certain embodiments, the mutation is F465Y. In certain embodiments, the mutation is T470V. In certain embodiments, the mutation is T470Y. In certain embodiments, the mutation is S473F. In certain embodiments, the mutation is P478L. In certain embodiments, the mutation is L486S.

In certain embodiments, the replication capacity of the HIV is increased. In other embodiments, the replication capacity of the HIV is decreased.

In other embodiments, the invention provides a method for determining that an HIV has altered replication capacity that comprises detecting a mutation in a codon of the region of pol that encodes RT that is selected from the group consisting of codons 39, 121, 135, 138, 196, 203, 204, 207, 210, 211, 245, 248, 275, 276, and 286, or any combination thereof. In certain embodiments, the mutation is in RT codon 39. In certain embodiments, the mutation is in RT codon 121. In certain embodiments, the mutation is in RT codon 135. In certain embodiments, the mutation is in RT codon 138. In certain embodiments, the mutation is in RT codon 196. In certain embodiments, the mutation is in RT codon 203. In certain embodiments, the mutation is in RT codon 204. In certain embodiments, the mutation is in RT codon 207. In certain embodiments, the mutation is in RT codon 210. In certain embodiments, the mutation is in RT codon 211. In certain embodiments, the mutation is in RT codon 245. In certain embodiments, the mutation is in RT codon 248. In certain embodiments, the mutation is in RT codon 275. In certain embodiments, the mutation is in RT codon 275. In certain embodiments, the mutation is in RT codon 276. In certain embodiments, the mutation is in RT codon 286.

In certain embodiments, the mutation can be selected from the group consisting of D121Y, I135V, E138A, G196E, E203D, E204D, E204K, Q207E, R211Q, V245E, E248D, K275Q, V276T, and T286P, or any combination thereof In certain embodiments, the mutation in RT is D121Y. In certain embodiments, the mutation in RT is I135V. In certain embodiments, the mutation in RT is E138A. In certain embodiments, the mutation in RT is G196E. In certain embodiments, the mutation in RT is E203D. In certain embodiments, the mutation in RT is E204D. In certain embodiments, the mutation in RT is E204K. In certain embodiments, the mutation in RT is E204K. In certain embodiments, the mutation in RT is Q207E. In certain embodiments, the mutation in RT is R211Q. In certain embodiments, the mutation in RT is V245E. In certain embodiments, the mutation in RT is E248D. In certain embodiments, the mutation in RT is K275Q. In certain embodiments, the mutation in RT is V276T. In certain embodiments, the mutation in RT is T286P.

In certain embodiments, the replication capacity of the HIV is increased. In other embodiments, the replication capacity of the HIV is decreased.

In still other embodiments, the invention provides a method for determining that an HIV has altered replication capacity that comprises detecting a mutation in a codon of the region of pol that encodes PR that is selected from the group consisting of codons 10, 14, 15, 20, 36, 37, 39, 61, 63, 64, 71, 72, 77, and 93, or any combination thereof. In certain embodiments, the mutation is in PR codon 10. In certain embodiments, the mutation is in PR codon 14. In certain embodiments, the mutation is in PR codon 15. In certain embodiments, the mutation is in PR codon 20. In certain embodiments, the mutation is in PR codon 36. In certain embodiments, the mutation is in PR codon 37. In certain embodiments, the mutation is in PR codon 39. In certain embodiments, the mutation is in PR codon 61. In certain embodiments, the mutation is in PR codon 63. In certain embodiments, the mutation is in PR codon 64. In certain embodiments, the mutation is in PR codon 71. In certain embodiments, the mutation is in PR codon 72. In certain embodiments, the mutation is in PR codon 77. In certain embodiments, the mutation is in PR codon 93.

In certain embodiments, the mutation can be selected from the group consisting of I15V, K20M, M36L, N37D, P39Q, P39S, Q61N, A71T, and V77I, or any combination thereof. In certain embodiments, the mutation is I15V. In certain embodiments, the mutation is K20M. In certain embodiments, the mutation is M36L. In certain embodiments, the mutation is N37D. In certain embodiments, the mutation is P39Q. In certain embodiments, the mutation is P39S. In certain embodiments, the mutation is Q61N. In certain embodiments, the mutation is A71T. In certain embodiments, the mutation is V77I. In certain embodiments, the replication capacity of the HIV is increased. In other embodiments, the replication capacity of the HIV is decreased.

In yet other embodiments, the invention provides a method for determining that an HIV has low replication capacity that comprises detecting a mutation in a codon of gag that is selected from the group consisting of 437, 439, 441, 442, 454, 478, 479, and 484, or any combination thereof. In certain embodiments, the mutation is in gag codon 437. In certain embodiments, the mutation is in gag codon 439. In certain embodiments, the mutation is in gag codon 441. In certain embodiments, the mutation is in gag codon 441. In certain embodiments, the mutation is in gag codon 442. In certain embodiments, the mutation is in gag codon 454. In certain embodiments, the mutation is in gag codon 478. In certain embodiments, the mutation is in gag codon 479. In certain embodiments, the mutation is in gag codon 484.

In certain embodiments, the mutation can be selected from the group consisting of I437L, P439S, E454V, P478L, and I479K, or any combination thereof. In certain embodiments, the mutation is I437L. In certain embodiments, the mutation is P439S. In certain embodiments, the mutation is E454V. In certain embodiments, the mutation is P478L. In certain embodiments, the mutation is I479K.

In still other embodiments, the invention provides a method for determining that an HIV has an altered replication capacity that comprises detecting a mutation in a codon of gag that is selected from the group consisting of 418, 456, 453, 483, 429, 484, 481, 465, 454, 442, 479, and 486, or any combination thereof. In certain embodiments, the mutation is in gag codon 418. In certain embodiments, the mutation is in gag codon 456. In certain embodiments, the mutation is in gag codon 453. In certain embodiments, the mutation is in gag codon 483. In certain embodiments, the mutation is in gag codon 481. In certain embodiments, the mutation is in gag codon 465. In certain embodiments, the mutation is in gag codon 429. In certain embodiments, the mutation is in gag codon 484. In certain embodiments, the mutation is in gag codon 454. In certain embodiments, the mutation is in gag codon 442. In certain embodiments, the mutation is in gag codon 479. In certain embodiments, the mutation is in gag codon 486.

In certain embodiments, the replication capacity of the HIV is increased. In other embodiments, the replication capacity of the HIV is decreased.

In certain embodiments, the methods comprise detecting a mutation in gag that is K418R, T456X, T456S, P453X, K418X, L483X, K481X, F465X, R429X, Y484X, K481R, L483-, Y484-, F465C, E454X, K442X, I479K, K418R, I479X, or L486S, or any combination thereof. In certain embodiments, the gag mutation is K418R. In certain embodiments, the gag mutation is T456X. In certain embodiments, the gag mutation is T456S. In certain embodiments, the gag mutation is P453X. In certain embodiments, the gag mutation is K418X. In certain embodiments, the gag mutation is L483X. In certain embodiments, the gag mutation is K481X. In certain embodiments, the gag mutation is F465X. In certain embodiments, the gag mutation is R429X. In certain embodiments, the gag mutation is Y484X. In certain embodiments, the gag mutation is Y484X. In certain embodiments, the gag mutation is K481R. In certain embodiments, the gag mutation is L483-. In certain embodiments, the gag mutation is L483-. In certain embodiments, the gag mutation is Y484-. In certain embodiments, the gag mutation is F465C. In certain embodiments, the gag mutation is E454X. In certain embodiments, the gag mutation is K442X. In certain embodiments, the gag mutation is K442X. In certain embodiments, the gag mutation is I479K. In certain embodiments, the gag mutation is I479K. In certain embodiments, the gag mutation is K418R. In certain embodiments, the gag mutation is I479X. In certain embodiments, the gag mutation is L486S.

In still other embodiments, the invention provides a method for determining that an HIV has an decreased replication capacity that comprises detecting a mutation in a codon of gag that is selected from the group consisting of codons 418, 427, 429, 437, 441, 442, 453, 456, 473, 477, 478, 481, 482, 483, 484, 486, and 487. In certain embodiments, the codon of gag is codon 418. In certain embodiments, the codon of gag is codon 427. In certain embodiments, the codon of gag is codon 429. In certain embodiments, the codon of gag is codon 437. In certain embodiments, the codon of gag is codon 441. In certain embodiments, the codon of gag is codon 442. In certain embodiments, the codon of gag is codon 453. In certain embodiments, the codon of gag is codon 456. In certain embodiments, the codon of gag is codon 473. In certain embodiments, the codon of gag is codon 477. In certain embodiments, the codon of gag is codon 478. In certain embodiments, the codon of gag is codon 481. In certain embodiments, the codon of gag is codon 482. In certain embodiments, the codon of gag is codon 483. In certain embodiments, the codon of gag is codon 484. In certain embodiments, the codon of gag is codon 486. In certain embodiments, the codon of gag is codon 487.

In certain embodiments, the gag mutation is K418R, T427P, R429G/K, I437L, H441C/G, K442G, P453S, T456S, S473P, E477G, P478L, K481R, E482D, Y484P, L486S, or A487I. In certain embodiments, the gag mutation is K418R. In certain embodiments, the gag mutation is T427P. In certain embodiments, the gag mutation is R429G. In certain embodiments, the gag mutation is R429K. In certain embodiments, the gag mutation is I437L. In certain embodiments, the gag mutation is H441C. In certain embodiments, the gag mutation is H441G. In certain embodiments, the gag mutation is K442G. In certain embodiments, the gag mutation is P453S. In certain embodiments, the gag mutation is T456S. In certain embodiments, the gag mutation is S473P. In certain embodiments, the gag mutation is E477G. In certain embodiments, the gag mutation is P478L. In certain embodiments, the gag mutation is P478L. In certain embodiments, the gag mutation is K481R. In certain embodiments, the gag mutation is E482D. In certain embodiments, the gag mutation is Y484P. In certain embodiments, the gag mutation is L486S. In certain embodiments, the gag mutation is A487I.

In still other embodiments, the invention provides a method for determining that an HIV has an increased replication capacity that comprises detecting a mutation in a codon of gag that is selected from the group consisting of codons 418, 465, 478, 479, 481, 482, 483, 484, and 486. In certain embodiments, the gag mutation is K418R, F465Y, P478L/Q, I479R, K481x, E482D, L483x, Y484x, or L486S.

In certain embodiments, the HIV with low replication capacity has a replication capacity that is decreased about 10%, about 20%, about 30%, about 40%, about 50%, about 60%, about 70%, about 80% about 90%, or about 95% relative to a reference HIV. In certain embodiments, the HIV with altered replication capacity has a replication capacity that is decreased about 10%, about 20%, about 30%, about 40%, about 50%, about 60%, about 70%, about 80% about 90%, or about 95% relative to a reference HIV. In other embodiments, the HIV with altered replication capacity has a replication capacity that is increased about 10%, about 20%, about 30%, about 40%, about 50%, about 60%, about 70%, about 80% about 90%, about 100%, about 110%, about 120%, about 130%, about 140%, about 150%, about 160%, about 170%, about 180% about 190%, about 200%, about 210%, about 220%, about 230%, about 240%, about 250%, about 260%, about 270%, about 280% about 290%, or about 300% relative to a reference HIV. In certain embodiments, the reference HIV is NL4-3.

In yet other embodiments, the invention provides a method for determining that an HIV has an altered replication capacity that comprises detecting any mutation in any HIV gene disclosed herein as correlated with altered replication capacity, thereby determining that the HIV's replication capacity is altered relative to a reference HIV. In preferred embodiments, the correlation between the mutation and altered replication capacity of the HIV is significant. In certain embodiments, the replication capacity is increased. In certain embodiments, the replication capacity is decreased. In certain embodiments, the reference virus is HIV strain NL4-3.

5.6. Computer-Implemented Methods and Articles Related thereto

In another aspect, the present invention provides computer-implemented methods for identifying a target for antiviral therapy. In such embodiments, the methods of the invention are adapted to take advantage of the processing power of modem computers. One of skill in the art can readily adapt the methods in such a manner.

Therefore, in certain embodiments, the invention provides a computer-implemented method for identifying a target for antiviral therapy that comprises inputting the replication capacity of a statistically significant number of individual viruses and the genotypes of a gene of said statistically significant number of viruses into a computer system, and determining with said computer system a correlation between said replication capacities and said genotypes of said gene, thereby identifying a target for antiviral therapy.

In another aspect, the invention provides a computer-implemented method for determining the replication capacity of a virus that comprises performing a method of the invention with a computer adapted to perform the method. In certain embodiments, the method is a method for determining the replication capacity of the virus. In certain embodiments, the method is a method for determining that a virus has an altered replication capacity. In certain embodiments, the replication capacity of the virus is increased. In certain embodiments, the replication capacity of the virus is decreased.

In certain embodiments, the method further comprises the step of displaying a correlation between a replication capacity and a genotype on a computer display. In other embodiments, the method further comprises the step of printing a correlation between a replication capacity and a genotype onto a tangible medium, such as, for example, paper.

In another aspect, the invention provides a printout of a correlation between a replication capacity and a genotype determined according to a method of the invention.

In still another aspect, the invention provides an article of manufacture that comprises computer-readable instructions for performing a method of the invention. In certain embodiments, the article is a random access memory. In certain embodiments, the article is a flash memory. In certain embodiments, the article is a fixed disk drive. In certain embodiments, the article is a floppy drive.

In yet another aspect, the invention provides a computer system that is configured to perform a method of the invention.

5.7. Methods of Identifying Compounds with Anti-HIV Activity

In yet another aspect, the invention provides methods for identifying compounds with anti-HIV activity. The methods generally rely on modulating or otherwise disrupting an interaction among viral molecules or between viral molecules and host cell molecules that is identified according to a method of the invention.

Thus, in certain embodiments, the invention provides a method for identifying a compound to be further evaluated for anti-HIV activity that comprises determining a replication capacity for an HIV in the presence and in the absence of the compound to be evaluated. In certain embodiments, the compound modulates a target identified according to a method of the invention. The virus is preferably HIV. The compound to be further evaluated for anti-HIV activity can be identified if the replication capacity of the HIV is lower in the presence of the compound than it is in the absence of the compound.

In other embodiments, the invention provides a method for identifying a compound with anti-HIV activity, that comprises determining a replication capacity for an HIV in the presence and in the absence of the compound to be evaluated. In certain embodiments, the compound modulates a target identified according to a method of the invention. The virus is preferably HIV. The compound with anti-HIV activity can be identified if the replication capacity of the HIV is lower in the presence of the compound than in the absence of the compound.

5.8. Viruses and Viral Samples

An altered replication capacity-associated mutation according to the present invention can be present in any type of virus. For example, such mutations may be identified in any virus that infects animals known to one skill in the art without limitation. In one embodiment of the invention, the virus includes viruses known to infect mammals, including dogs, cats, horses, sheep, cows etc. In certain embodiment, the virus is known to infect primates. In preferred embodiments, the virus is known to infect humans. Examples of such viruses that infect humans include, but are not limited to, human immunodeficiency virus (“HIV”), herpes simplex virus, cytomegalovirus virus, varicella zoster virus, other human herpes viruses, influenza A, B and C virus, respiratory syncytial virus, hepatitis A, B and C viruses, rhinovirus, and human papilloma virus. In certain embodiments, the virus is HCV. In other embodiments, the virus is HBV. In a preferred embodiment of the invention, the virus is HIV. Even more preferably, the virus is human immunodeficiency virus type 1 (“HIV-1”). The foregoing are representative of certain viruses for which there is presently available anti-viral chemotherapy and represent the viral families retroviridae, herpesviridae, orthomyxoviridae, paramxyxoviridae, picomaviridae, flaviviridae, pneumoviridae and hepadnaviridae. This invention can be used with other viral infections due to other viruses within these families as well as viral infections arising from viruses in other viral families for which there is or there is not a currently available therapy.

An altered replication capacity-associated mutation according to the present invention can be found in a viral sample obtained by any means known in the art for obtaining viral samples. Such methods include, but are not limited to, obtaining a viral sample from a human or an animal infected with the virus or obtaining a viral sample from a viral culture. In one embodiment, the viral sample is obtained from a human individual infected with the virus. The viral sample could be obtained from any part of the infected individual's body or any secretion expected to contain the virus. Examples of such parts include, but are not limited to blood, serum, plasma, sputum, lymphatic fluid, semen, vaginal mucus and samples of other bodily fluids. In a preferred embodiment, the sample is a blood, serum or plasma sample.

In another embodiment, an altered replication capacity-associated mutation according to the present invention is present in a virus that can be obtained from a culture. In some embodiments, the culture can be obtained from a laboratory. In other embodiments, the culture can be obtained from a collection, for example, the American Type Culture Collection.

In certain embodiments, an altered replication capacity-associated mutation according to the present invention is present in a derivative of a virus. In one embodiment, the derivative of the virus is not itself pathogenic. In another embodiment, the derivative of the virus is a plasmid-based system, wherein replication of the plasmid or of a cell transfected with the plasmid is affected by the presence or absence of the selective pressure, such that mutations are selected that increase resistance to the selective pressure. In some embodiments, the derivative of the virus comprises the nucleic acids or proteins of interest, for example, those nucleic acids or proteins to be targeted by an anti-viral treatment. In one embodiment, the genes of interest can be incorporated into a vector. See, e.g., U.S. Pat. Nos. 5,837,464 and 6,242,187 and PCT publication, WO 99/67427, each of which is incorporated herein by reference. In certain embodiments, the genes can be those that encode for a protease or reverse transcriptase.

In another embodiment, the intact virus need not be used. Instead, a part of the virus incorporated into a vector can be used. Preferably that part of the virus is used that is targeted by an anti-viral drug.

In another embodiment, an altered replication capacity-associated mutation according to the present invention is present in a genetically modified virus. The virus can be genetically modified using any method known in the art for genetically modifying a virus. For example, the virus can be grown for a desired number of generations in a laboratory culture. In one embodiment, no selective pressure is applied (i.e., the virus is not subjected to a treatment that favors the replication of viruses with certain characteristics), and new mutations accumulate through random genetic drift. In another embodiment, a selective pressure is applied to the virus as it is grown in culture (i.e., the virus is grown under conditions that favor the replication of viruses having one or more characteristics). In one embodiment, the selective pressure is an anti-viral treatment. Any known anti-viral treatment can be used as the selective pressure.

In certain embodiments, the virus is HIV and the selective pressure is a NNRTI. In another embodiment, the virus is HIV-1 and the selective pressure is a NNRTI. Any NNRTI can be used to apply the selective pressure. Examples of NNRTIs include, but are not limited to, nevirapine, delavirdine and efavirenz. By treating HIV cultured in vitro with a NNRTI, one can select for mutant strains of HIV that have an increased resistance to the NNRTI. The stringency of the selective pressure can be manipulated to increase or decrease the survival of viruses not having the selected-for characteristic.

In other embodiments, the virus is HIV and the selective pressure is a NRTI. In another embodiment, the virus is HIV-1 and the selective pressure is a NRTI. Any NRTI can be used to apply the selective pressure. Examples of NRTIs include, but are not limited to, AZT, ddI, ddC, d4T, 3TC, and abacavir. By treating HIV cultured in vitro with a NRTI, one can select for mutant strains of HIV that have an increased resistance to the NRTI. The stringency of the selective pressure can be manipulated to increase or decrease the survival of viruses not having the selected-for characteristic.

In still other embodiments, the virus is HIV and the selective pressure is a PI. In another embodiment, the virus is HIV-1 and the selective pressure is a PI. Any PI can be used to apply the selective pressure. Examples of PIs include, but are not limited to, saquinavir, ritonavir, indinavir, nelfinavir, amprenavir, lopinavir and atazanavir. By treating HIV cultured in vitro with a PI, one can select for mutant strains of HIV that have an increased resistance to the PI. The stringency of the selective pressure can be manipulated to increase or decrease the survival of viruses not having the selected-for characteristic.

In still other embodiments, the virus is HIV and the selective pressure is an entry inhibitor. In another embodiment, the virus is HIV-1 and the selective pressure is an entry inhibitor. Any entry inhibitor can be used to apply the selective pressure. An example of a entry inhibitor includes, but is not limited to, fusion inhibitors such as, for example, enfuvirtide. Other entry inhibitors include co-receptor inhibitors, such as, for example, AMD3100 (Anormed). Such co-receptor inhibitors can include any compound that interferes with an interaction between HIV and a co-receptor, e.g., CCR5 or CRCX4, without limitation. By treating HIV cultured in vitro with an entry inhibitor, one can select for mutant strains of HIV that have an increased resistance to the entry inhibitor. The stringency of the selective pressure can be manipulated to increase or decrease the survival of viruses not having the selected-for characteristic.

In another aspect, an altered replication capacity-associated mutation according to the present invention is made by mutagenizing a virus, a viral genome, or a part of a viral genome. Any method of mutagenesis known in the art can be used for this purpose. In certain embodiments, the mutagenesis is essentially random. In certain embodiments, the essentially random mutagenesis is performed by exposing the virus, viral genome or part of the viral genome to a mutagenic treatment. In another embodiment, a gene that encodes a viral protein that is the target of an anti-viral therapy is mutagenized. Examples of essentially random mutagenic treatments include, for example, exposure to mutagenic substances (e.g., ethidium bromide, ethylmethanesulphonate, ethyl nitroso urea (ENU) etc.) radiation (e.g., ultraviolet light), the insertion and/or removal of transposable elements (e.g., Tn5, Tn10), or replication in a cell, cell extract, or in vitro replication system that has an increased rate of mutagenesis. See, e.g., Russell et al., 1979, Proc. Nat. Acad. Sci. USA 76:5918-5922; Russell, W., 1982, Environmental Mutagens and Carcinogens: Proceedings of the Third International Conference on Environmental Mutagens. One of skill in the art will appreciate that while each of these methods of mutagenesis is essentially random, at a molecular level, each has its own preferred targets.

In another aspect, an altered replication capacity-associated mutation is made using site-directed mutagenesis. Any method of site-directed mutagenesis known in the art can be used (see e.g., Sambrook et al., 2001, Molecular Cloning: A Laboratory Manual, Cold Spring Harbor Laboratory, 3^{rd} ed., NY; and Ausubel et al., 1989, Current Protocols in Molecular Biology, Greene Publishing Associates and Wiley Interscience, NY). The site directed mutagenesis can be directed to, e.g., a particular gene or genomic region, a particular part of a gene or genomic region, or one or a few particular nucleotides within a gene or genomic region. In one embodiment, the site directed mutagenesis is directed to a viral genomic region, gene, gene fragment, or nucleotide based on one or more criteria. In one embodiment, a gene or a portion of a gene is subjected to site-directed mutagenesis because it encodes a protein that is known or suspected to be a target of an anti-viral therapy, e.g., the gene encoding the HIV reverse transcriptase. In another embodiment, a portion of a gene, or one or a few nucleotides within a gene, are selected for site-directed mutagenesis. In one embodiment, the nucleotides to be mutagenized encode amino acid residues that are known or suspected to interact with an anti-viral compound. In another embodiment, the nucleotides to be mutagenized encode amino acid residues that are known or suspected to be mutated in viral strains having an altered replication capacity. In another embodiment, the mutagenized nucleotides encode amino acid residues that are adjacent to or near in the primary sequence of the protein residues known or suspected to interact with an anti-viral compound or known or suspected to be mutated in viral strains having an altered replication capacity. In another embodiment, the mutagenized nucleotides encode amino acid residues that are adjacent to or near to in the secondary, tertiary or quaternary structure of the protein residues known or suspected to interact with an anti-viral compound or known or suspected to be mutated in viral strains having an altered replication capacity. In another embodiment, the mutagenized nucleotides encode amino acid residues in or near the active site of a protein that is known or suspected to bind to an anti-viral compound. See, e.g., Sarkar and Sommer, 1990, Biotechniques, 8:404-407.

### 6. EXAMPLES

### 6.1. Example 1

### Measuring Replication Capacity Using Resistance Test Vectors

This example provides methods and compositions for accurately and reproducibly measuring the resistance or sensitivity of HIV-1 to antiretroviral drugs as well as the replication capacity of the HIV-1. The methods for measuring resistance or susceptibility to such drugs or replication capacity can be adapted to other HIV strains, such as HIV-2, or to other viruses, including, but not limited to hepadnaviruses (e.g., human hepatitis B virus), flaviviruses (e.g., human hepatitis C virus) and herpesviruses (e.g., human cytomegalovirus).

Replication capacity tests can be carried out using the methods for phenotypic drug susceptibility and resistance tests described in U.S. Pat. No. 5,837,464 (International Publication Number WO 97/27319) which is hereby incorporated by reference in its entirety, or according to the protocol that follows.

Patient-derived segment(s) corresponding to the HIV protease and reverse transcriptase coding regions were amplified by the reverse transcription-polymerase chain reaction method (RT-PCR) using viral RNA isolated from viral particles present in the plasma or serum of HIV-infected individuals as follows. Viral RNA was isolated from the plasma or serum using oligo-dT magnetic beads (Dynal Biotech, Oslo, Norway), followed by washing and elution of viral RNA. The RT-PCR protocol was divided into two steps. A retroviral reverse transcriptase (e.g. Moloney MuLV reverse transcriptase (Roche Molecular Systems, Inc., Branchburg, N.J.; Invitrogen, Carlsbad, Calif.), or avian myeloblastosis virus (AMV) reverse transcriptase, (Boehringer Mannheim, Indianapolis, Ind.), or) was used to copy viral RNA into cDNA. The cDNA was then amplified using a thermostable DNA polymerase (e.g. Taq (Roche Molecular Systems, Inc., Branchburg, N.J.), Tth (Roche Molecular Systems, Inc., Branchburg, N.J.), PRIMEZYME™ (isolated from Thermus brockianus, Biometra, Gottingen, Germany)) or a combination of thermostable polymerases as described for the performance of “long PCR” (Barnes, W. M., 1994, Proc. Natl. Acad. Sci, USA 91, 2216-20) (e.g. Expand High Fidelity PCR System (Taq+Pwo), (Boehringer Mannheim. Indianapolis, Ind.); GENEAMP XL™ PCR kit (Tth+Vent), (Roche Molecular Systems, Inc., Branchburg, N.J.); or ADVANTAGE II®, Clontech, Palo Alto, Calif.)

PCR primers were designed to introduce ApaI and PinA1 recognition sites into the 5′ or 3′ end of the PCR product, respectively.

Replication capacity test vectors incorporating the “test” patient-derived segments were constructed as described in U.S. Pat. No. 5,837,464 using an amplified DNA product of 1.5 kB prepared by RT-PCR using viral RNA as a template and oligonucleotides PDS Apa, PDS Age, PDS PCR6, Apa-gen, Apa-c, Apa-f, Age-gen, Age-a, RT-ad, RT-b, RT-c, RT-f, and/or RT-g as primers, followed by digestion with ApaI and AgeI or the isoschizomer PinA1. To ensure that the plasmid DNA corresponding to the resultant fitness test vector comprises a representative sanple of the HIV viral quasi-species present in the serum of a given patient, many (>250) independent E. coli transformants obtained in the construction of a given fitness test vector are pooled and used for the preparation of plasmid DNA.

A packaging expression vector encoding an amphotrophic MuLV 4070A env gene product enables production in a replication capacity test vector host cell of replication capacity test vector viral particles which can efficiently infect human target cells. Replication capacity test vectors encoding all HIV genes with the exception of env were used to transfect a packaging host cell (once transfected the host cell is referred to as a fitness test vector host cell). The packaging expression vector which encodes the amphotrophic MuLV 4070A env gene product is used with the replication capacity test vector to enable production in the replication capacity test vector host cell of infectious pseudotyped replication capacity test vector viral particles.

Replication capacity tests performed with resistance test vectors were carried out using packaging host and target host cells consisting of the human embryonic kidney cell line 293. Replication capacity tests were carried out with resistance test vectors using two host cell types. Resistance test vector viral particles were produced by a first host cell (the resistance test vector host cell) that was prepared by transfecting a packaging host cell with the resistance test vector and the packaging expression vector. The resistance test vector viral particles were then used to infect a second host cell (the target host cell) in which the expression of the indicator gene is measured.

The resistance test vectors containing a finctional luciferase gene cassette were constructed as described above and host cells were transfected with the resistance test vector DNA. The resistance test vectors contained patient-derived reverse transcriptase and protease DNA sequences that encode proteins which were either susceptible or resistant to the antiretroviral agents, such as, for example, NRTIs, NNRTIs, and PIs.

The amount of luciferase activity detected in infected cells is used as a direct measure of “infectivity,” i.e., the ability of the virus to complete a single round of replication. Thus, drug resistance or sensitivity can be determined by plotting the amount of luciferase activity produced by patient derived viruses in the presence of varying concentrations of the antiviral drug. By identifying the concentration of drug at which luciferase activity is half-maximum, the IC₅₀ of the virus from which patient-derived segment(s) were obtained for the antiretroviral agent can be determined. Alternatively, the amount of luciferase activity observed in the absence of any antiviral drug serves as a direct measure of the replication capacity of the virus.

Host (293) cells were seeded in 10-cm-diameter dishes and were transfected one day after plating with resistance test vector plasmid DNA and the envelope expression vector. Transfections were performed using a calcium-phosphate co-precipitation procedure. The cell culture media containing the DNA precipitate was replaced with fresh medium, from one to 24 hours, after transfection. Cell culture medium containing resistance test vector viral particles was harvested one to four days after transfection and was passed through a 0.45-mm filter before optional storage at −80° C. Before infection, target cells (293 cells) were plated in cell culture media. Control infections were performed using cell culture media from mock transfections (no DNA) or transfections containing the resistance test vector plasmid DNA without the envelope expression plasmid. One to three or more days after infection the media was removed and cell lysis buffer (Promega Corp.; Madison, Wis.) was added to each well. Cell lysates were assayed for luciferase activity. Alternatively, cells were lysed and luciferase was measured by adding Steady-Glo (Promega Corp.; Madison, Wis.) reagent directly to each well without aspirating the culture media from the well. The amount of luciferase activity produced in infected cells is normalized to adjust for variation in transfection efficiency in the transfected host cells by measuring the luciferase activity in the transfected cells, which is not dependent on viral gene functions, and adjusting the luciferase activity from infected cell accordingly.

### 6.2. Example 2

### Identifying Mutations Correlated with Altered Replication Fitness

This example provides methods and compositions for identifying mutations that correlate with altered replication fitness in the p6 gag protein or in HIV protease. The methods for identifying mutations that alter replication fitness can be adapted identify mutations in other components of HIV-1 replication, including, but not limited to, reverse transcription, integration, virus assembly, genome replication, virus attachment and entry, and any other essential phase of the viral life cycle. This example also provides a method for quantifying the effect that specific mutations in p6 gag protein and protease have on replication fitness. Means and methods for quantifying the effect that specific protease and reverse transcriptase mutations have on replication fitness can be adapted to mutations in other viral genes involved in HIV-1 replication, including, but not limited to the gag, pol, and env genes.

Replication capacity test vectors were constructed and used as described in Example 1. Replication capacity test vectors derived from patient samples or clones derived from the replication capacity test vector pools were tested in a replication capacity assay to determine accurately and quantitatively the relative replication capacity compared to the median observed replication capacity.

Genotypic Analysis of Patient HIV Samples:

Replication capacity test vector DNAs, either pools or clones, can be analyzed by any genotyping method, e.g., as described above. In this example, patient HIV sample sequences were determined using viral RNA purification, RT/PCR and ABI chain terminator automated sequencing. The sequence that was determined was compared to that of a reference sequence, NL4-3. The genotype was examined for sequences that were different from the reference or pre-treatment sequence and correlated to the observed replication capacity.

Correlation of Altered Replication Capacity and Mutations:

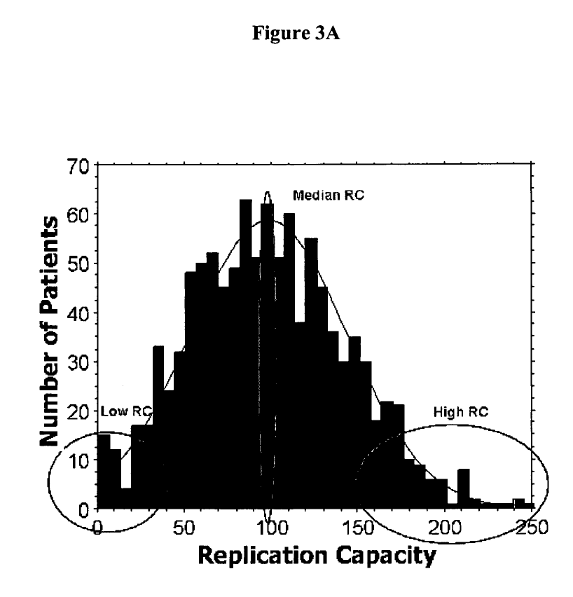

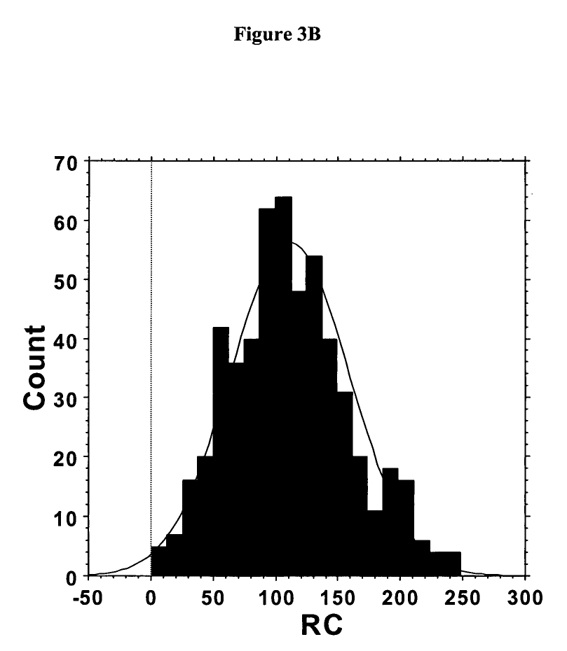

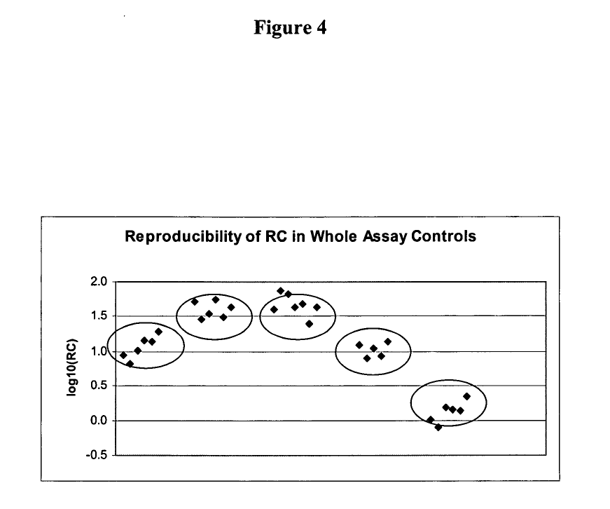

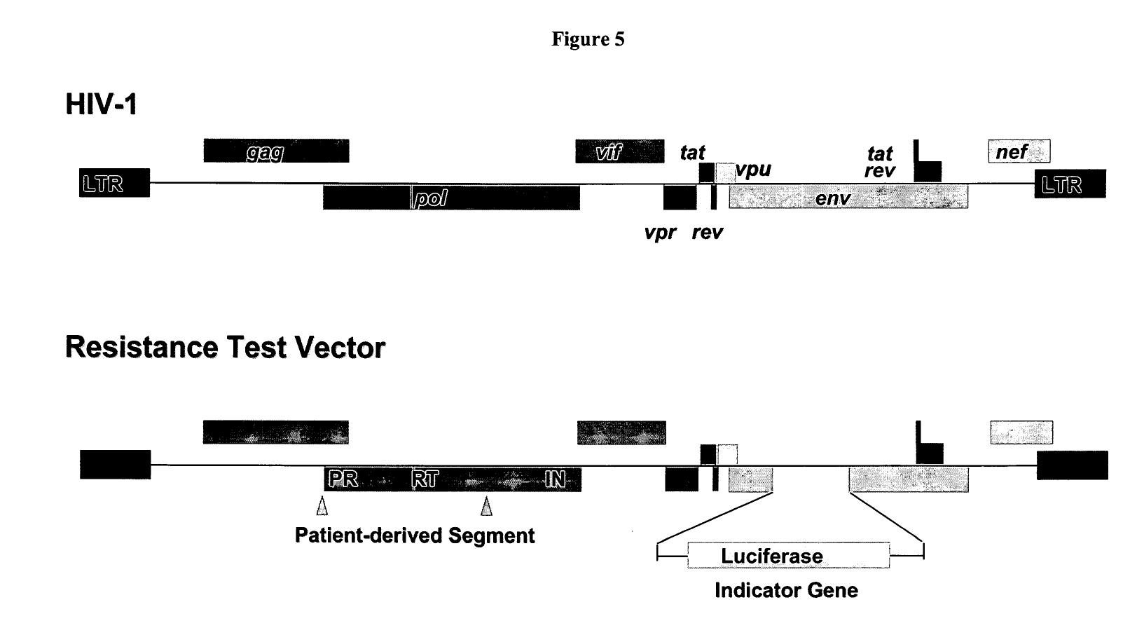

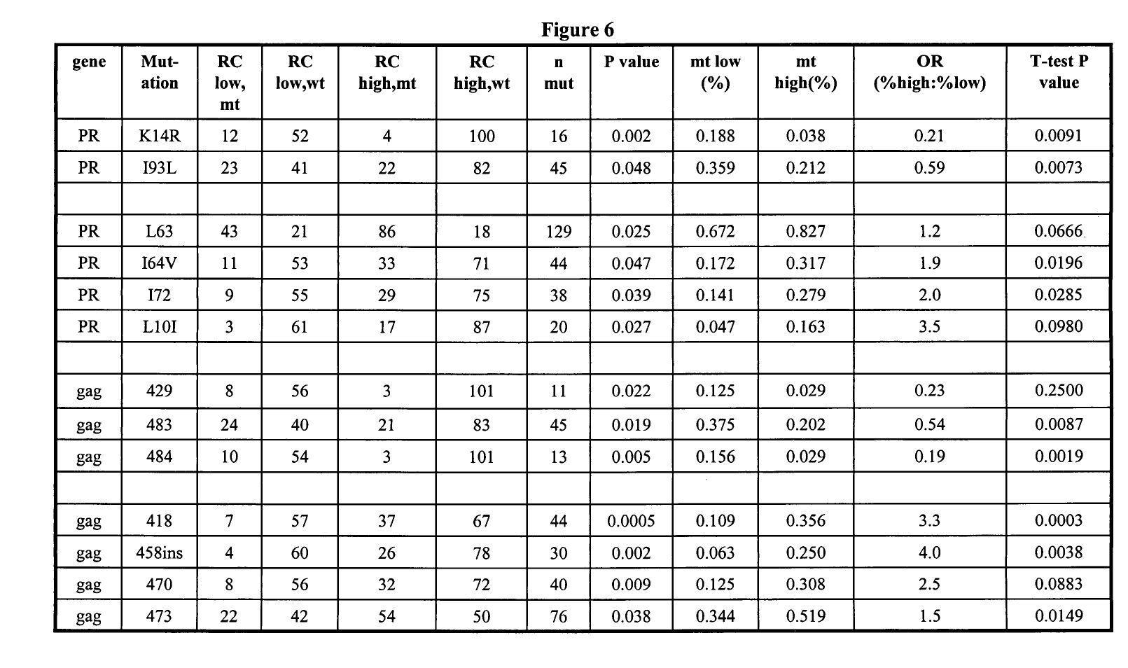

To identify mutations in gag, PR or RT associated with low or high replication capacity, two separate sets of analyses were performed. In the first set, from a collection of 1063 subtype B samples with RC values available and which had no known resistance-associated mutations in PR or RT (“wild-type” samples), 168 gag sequences were determined. The 168 samples were chosen based on their replication capacity falling in one of 3 different groups: below 37% (low, n=64), above 151% (high, n=80), or between 95 and 98% (medium, n=24). See FIG. 3A. Using an RC threshold of 50%, Fisher's Exact test was performed for each position in gag from 418 to 500 (the portion of gag that is contained in the PCR amplicon generated from the patient sample in PhenoSenseHIV). Mixtures of two or more amino acids at any position were ignored. All insertions close to the PTAP domain were considered as one variable termed “458ins” (the alignment method used for the gag sequences placed the insertions near PTAP after amino acid 458 of gag). All amino acid variants at each position in gag were considered together as one variable. Similarly, the same approach was used for PR, except that individual amino acids at each position were also considered separately. Results from this analysis are summarized in FIG. 6. In addition to Fisher's Exact test, the significance of the mutations identified above was further tested using the Student t-test for comparison of means using the Statview statistical software package (SAS, Cary N.C.) (see FIG. 6).

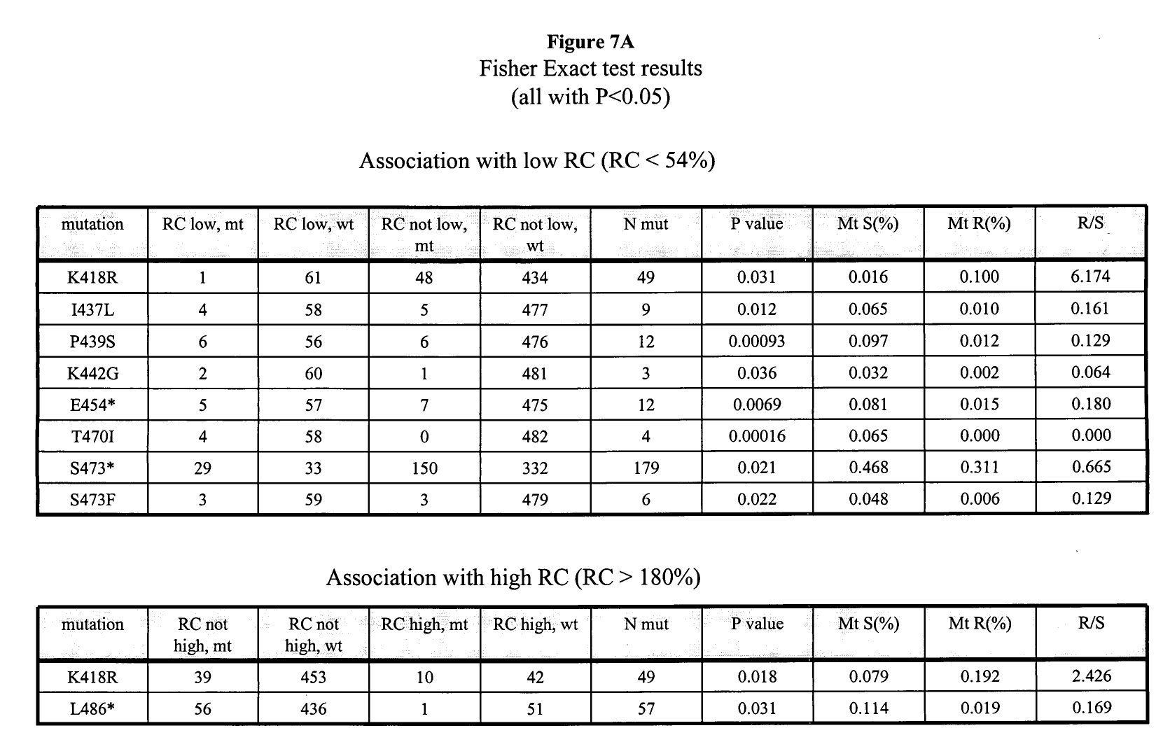

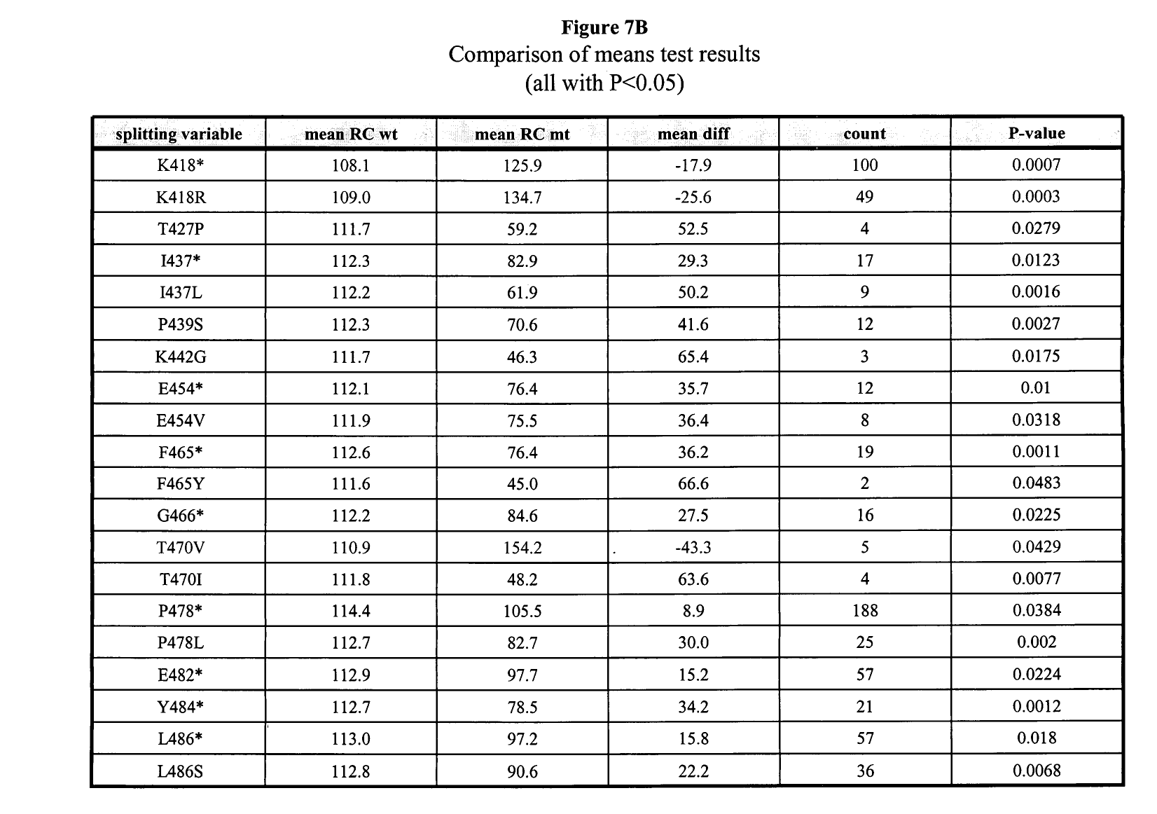

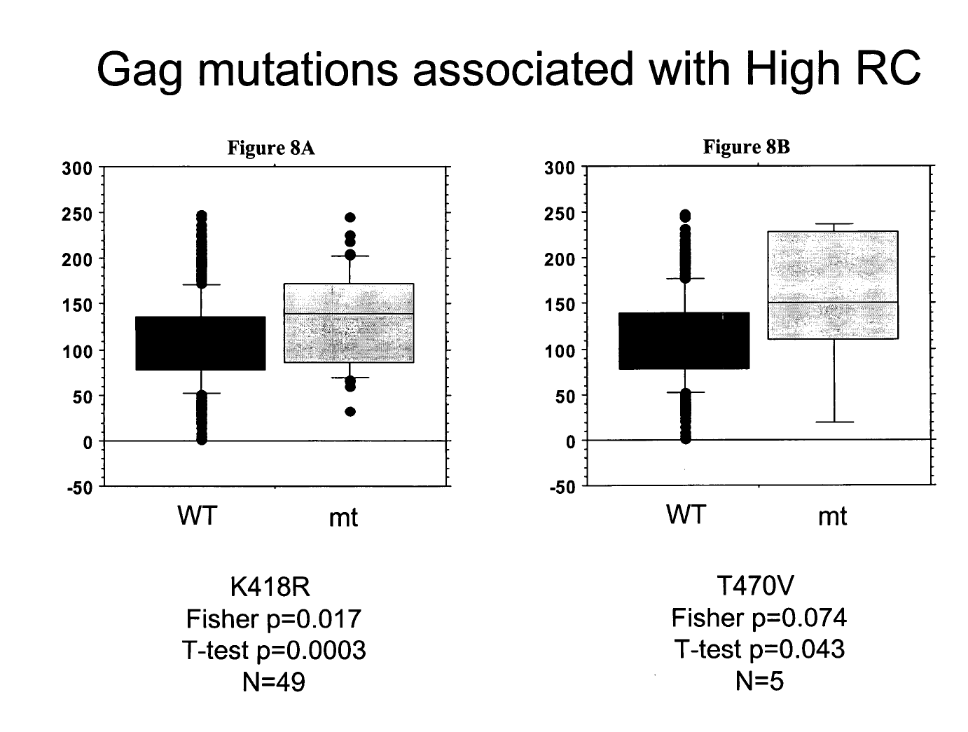

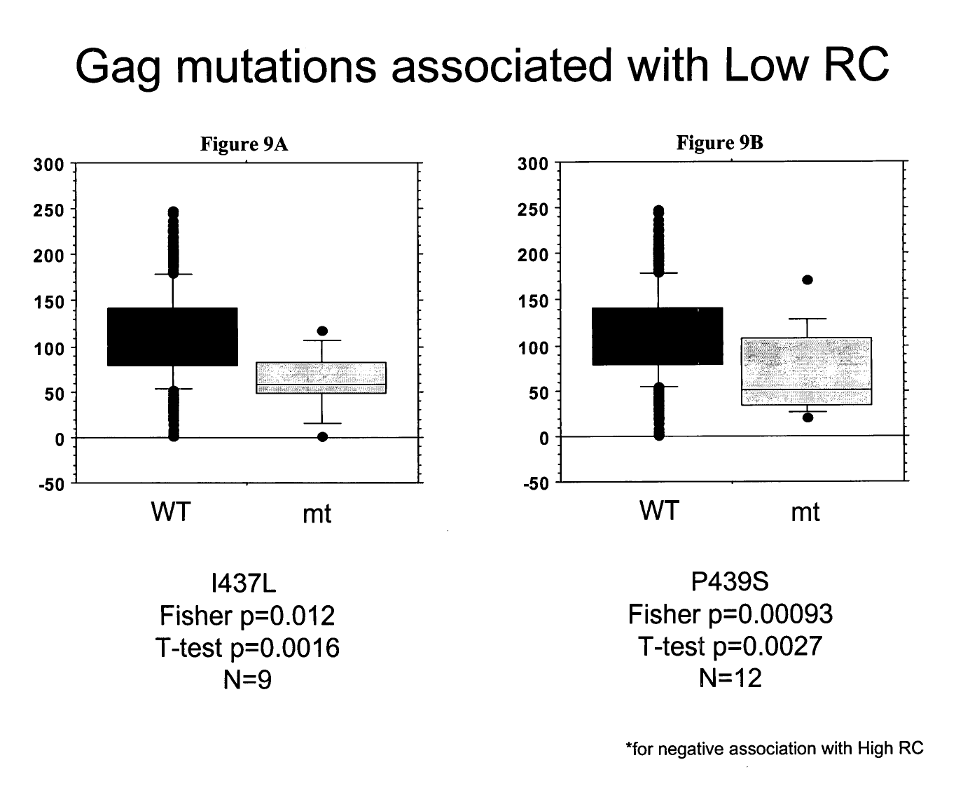

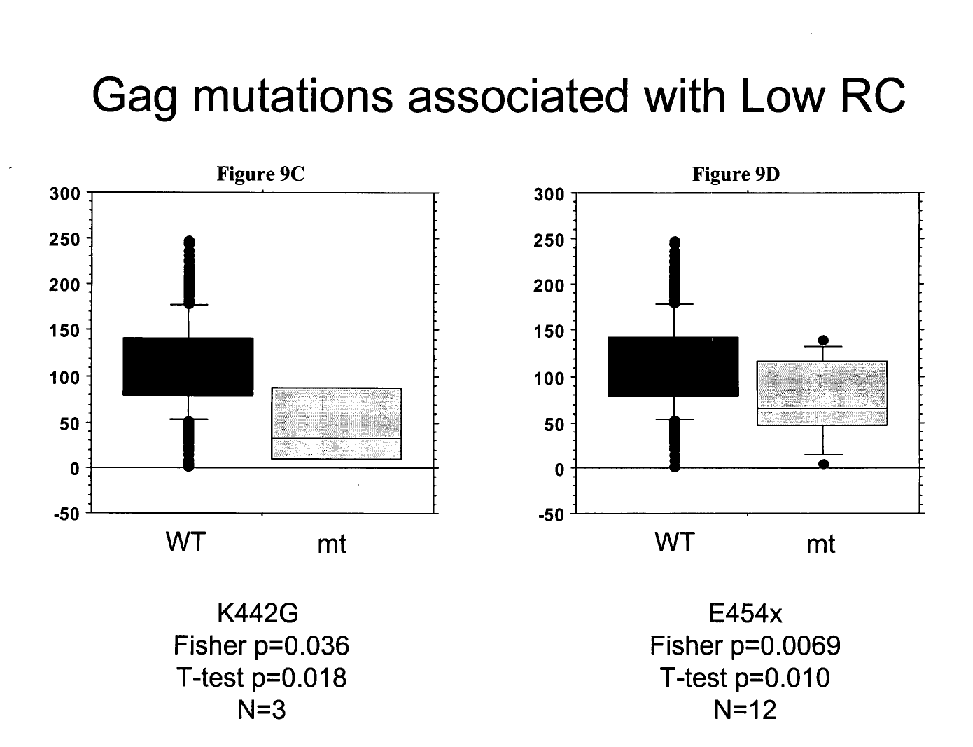

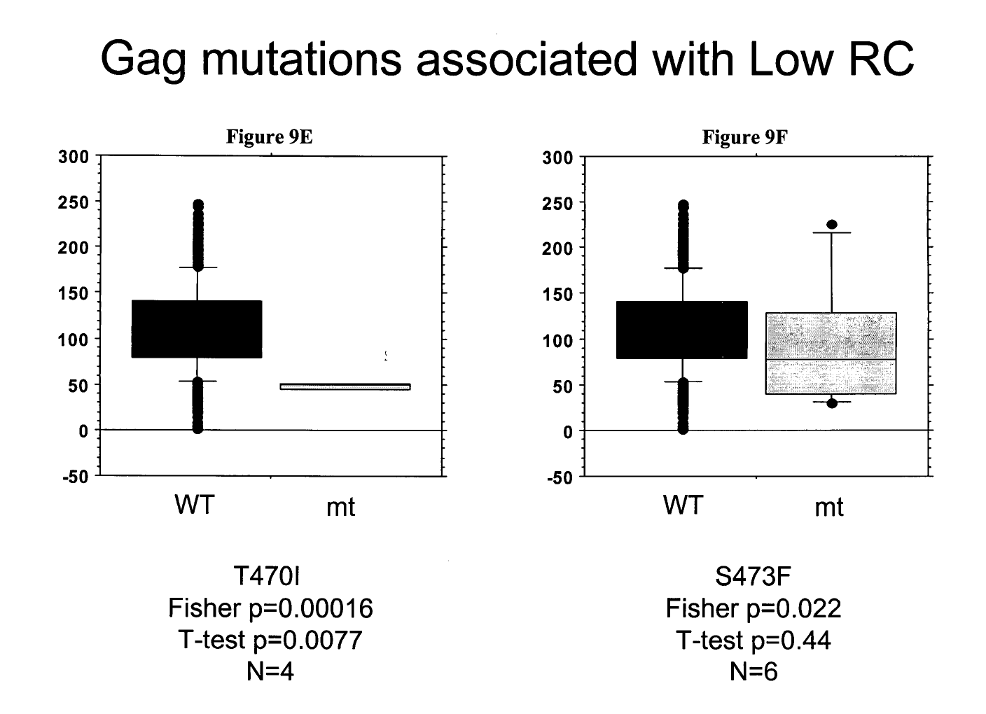

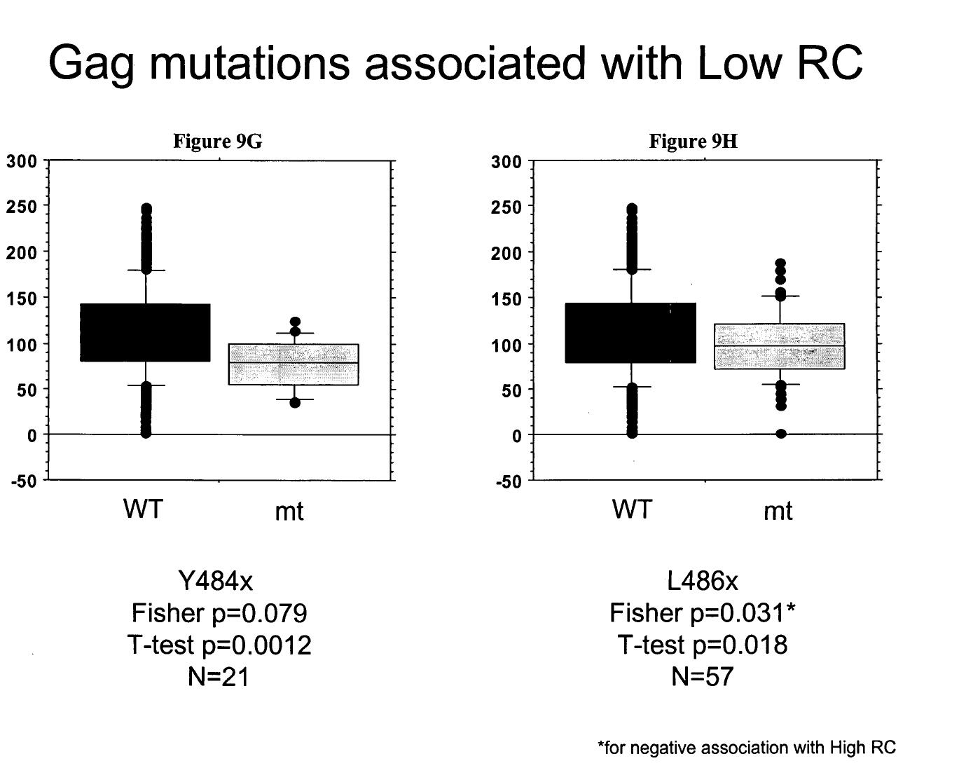

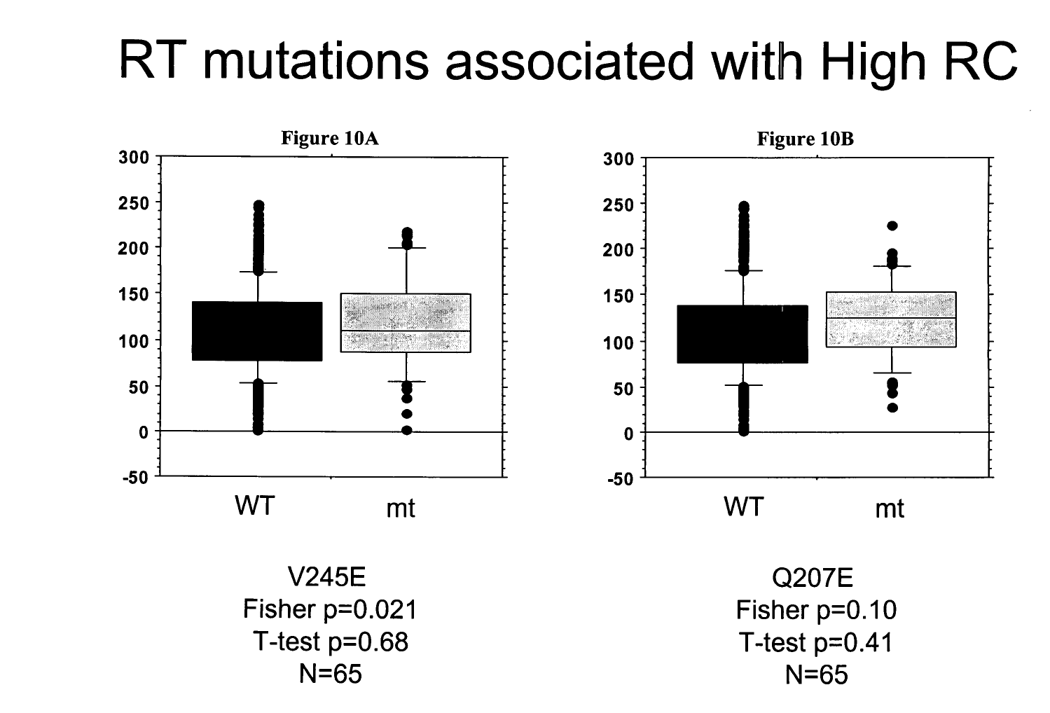

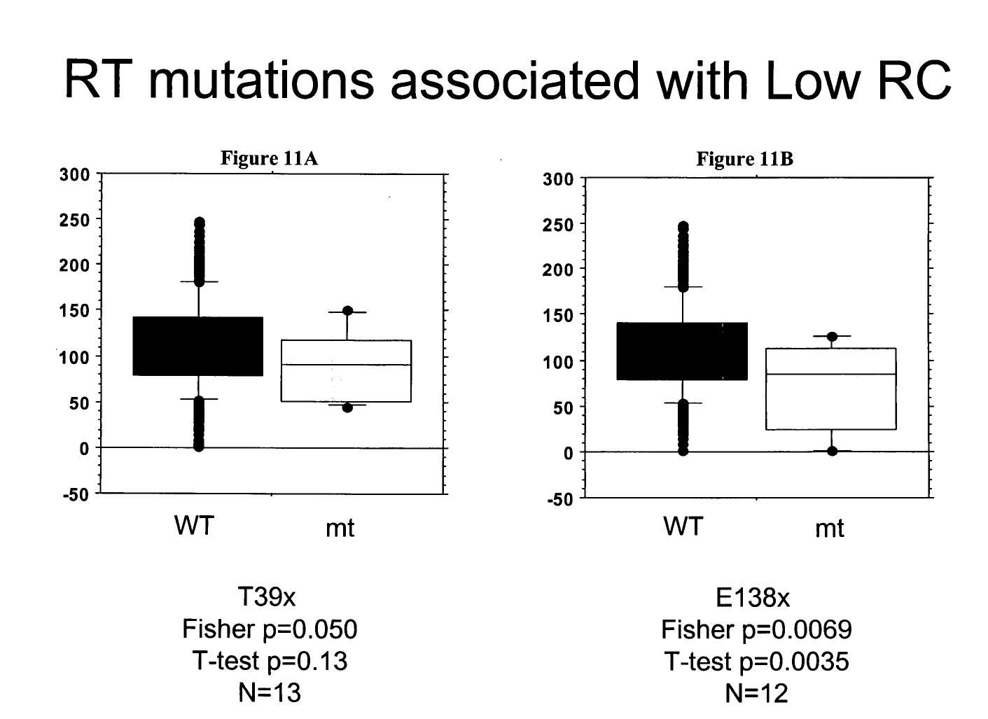

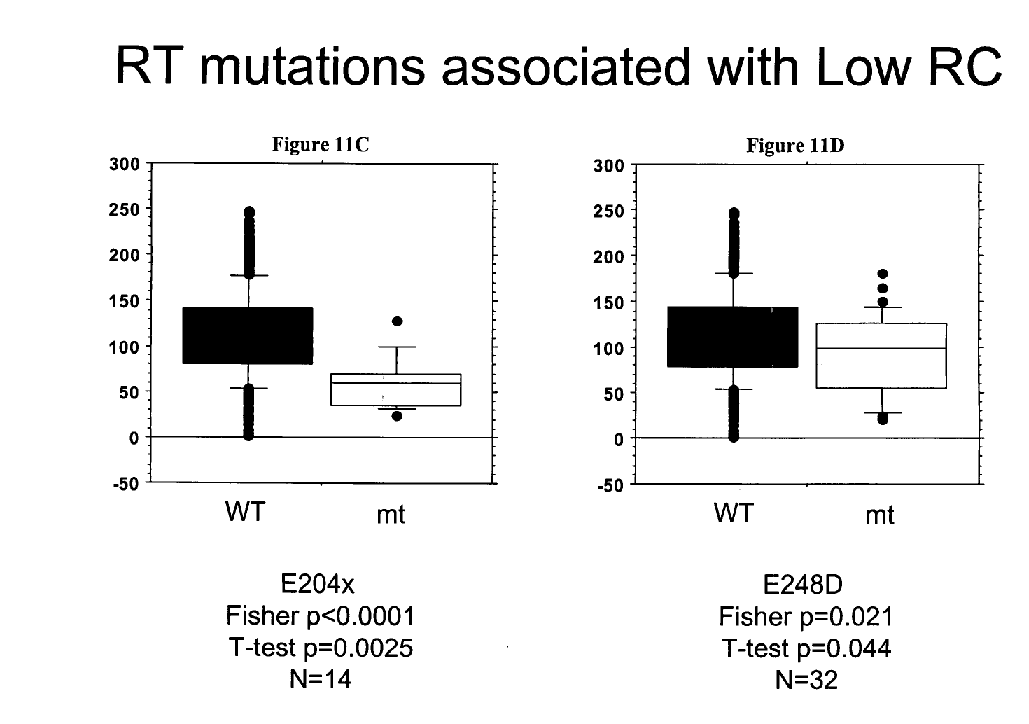

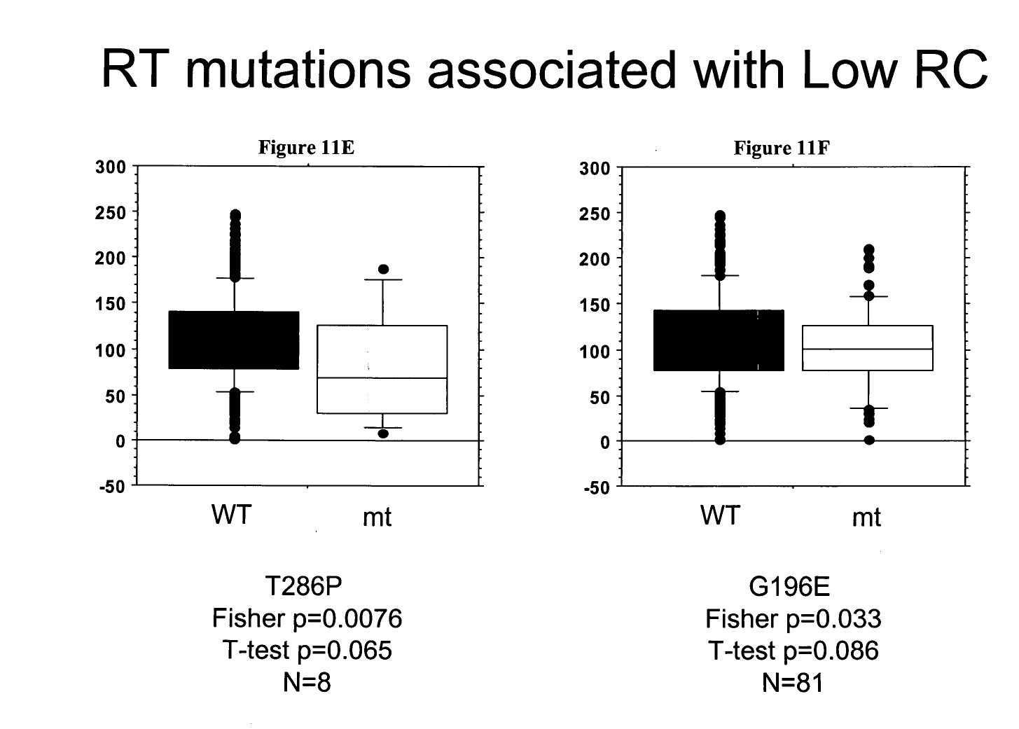

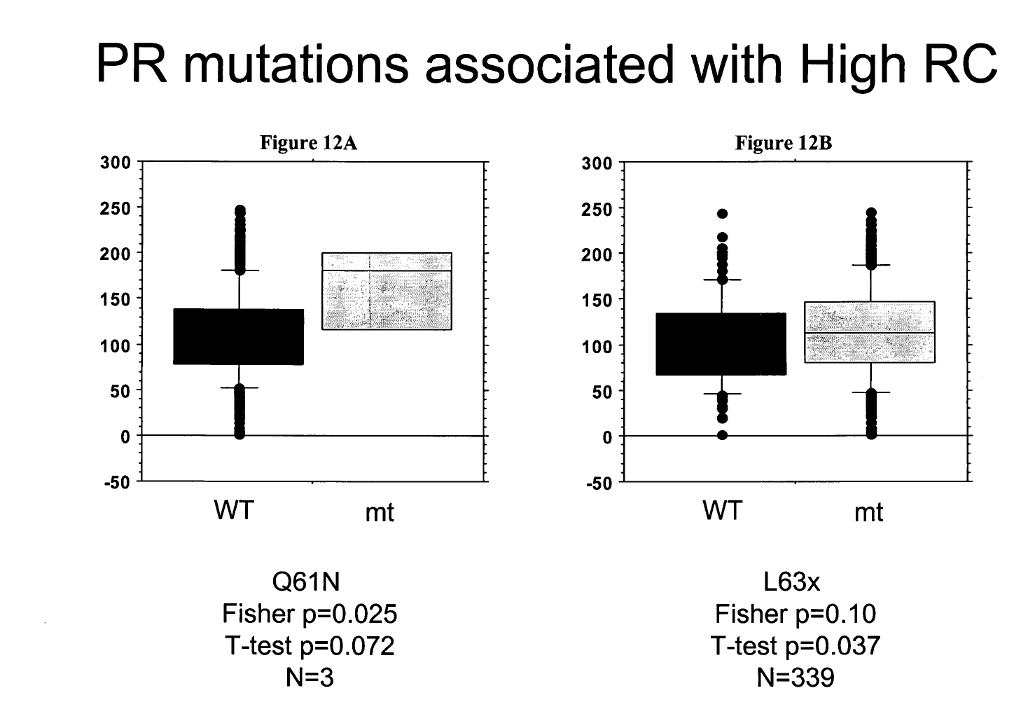

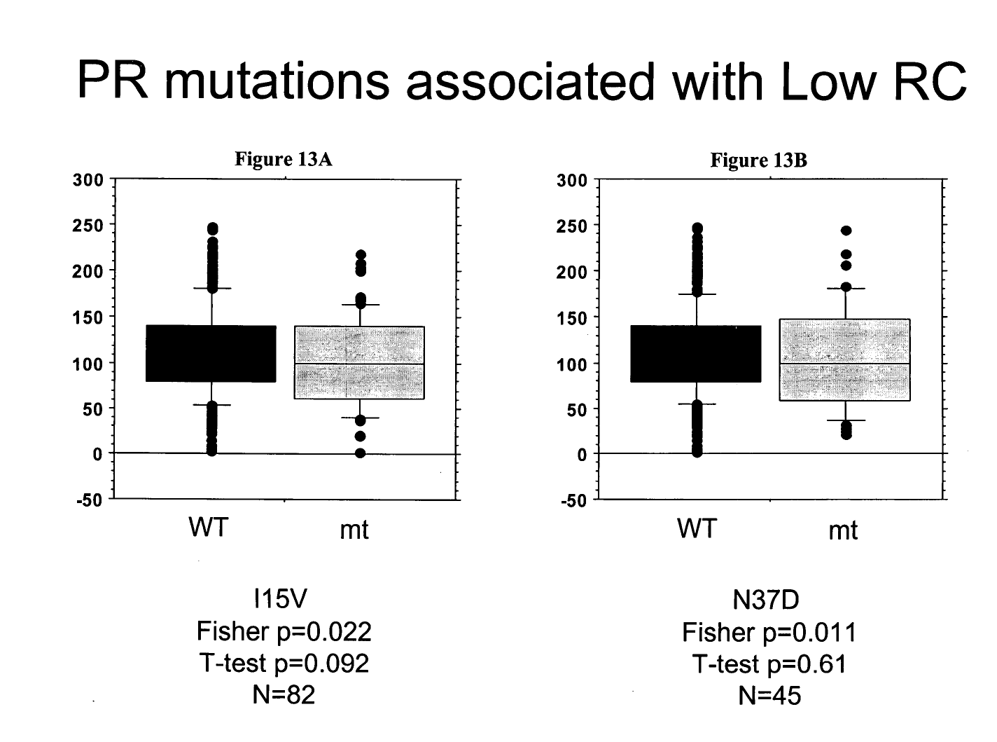

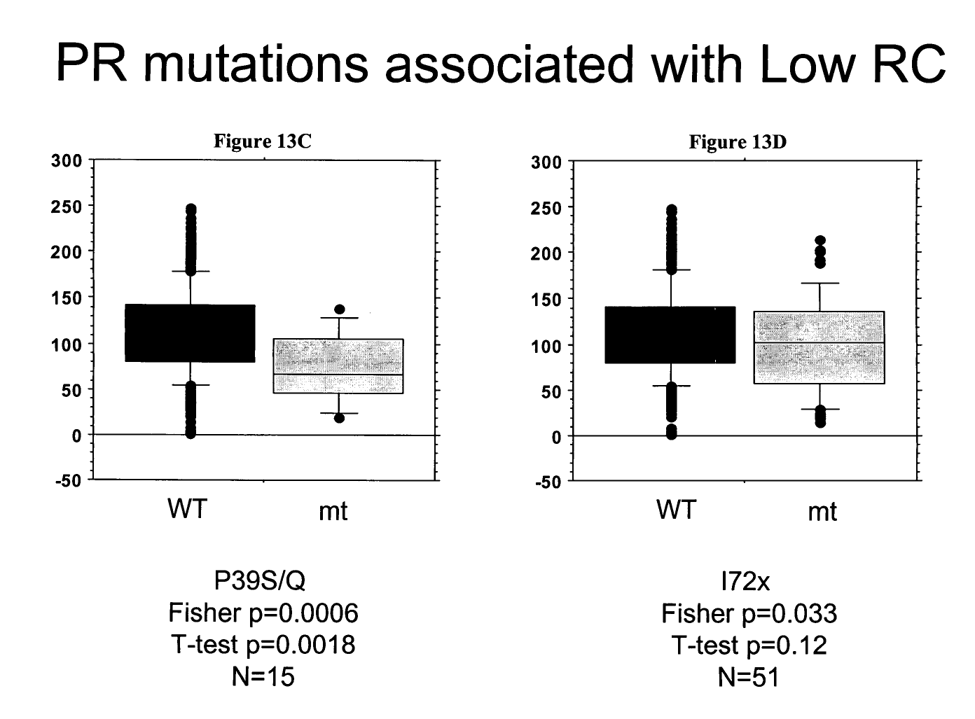

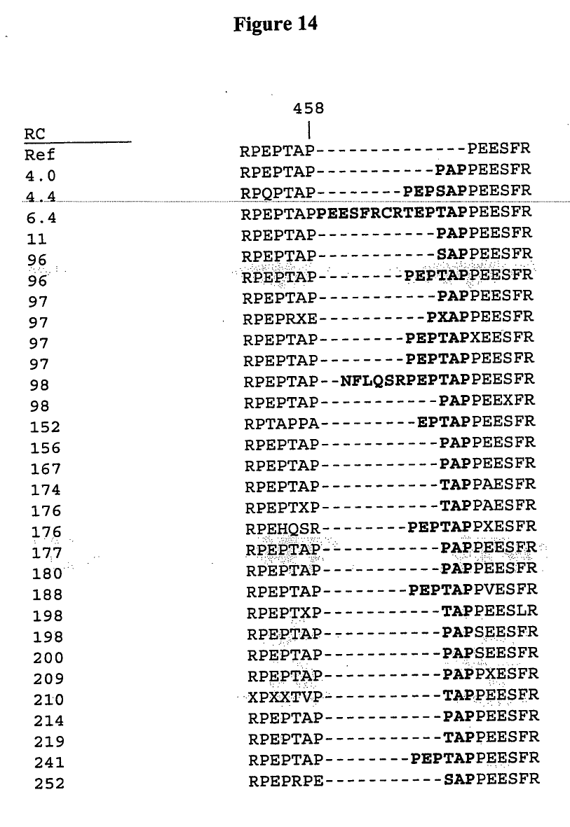

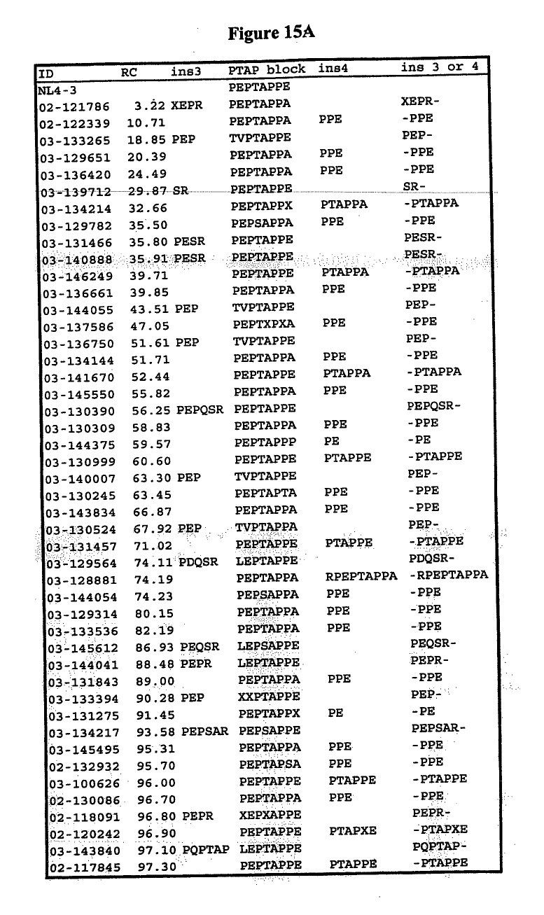

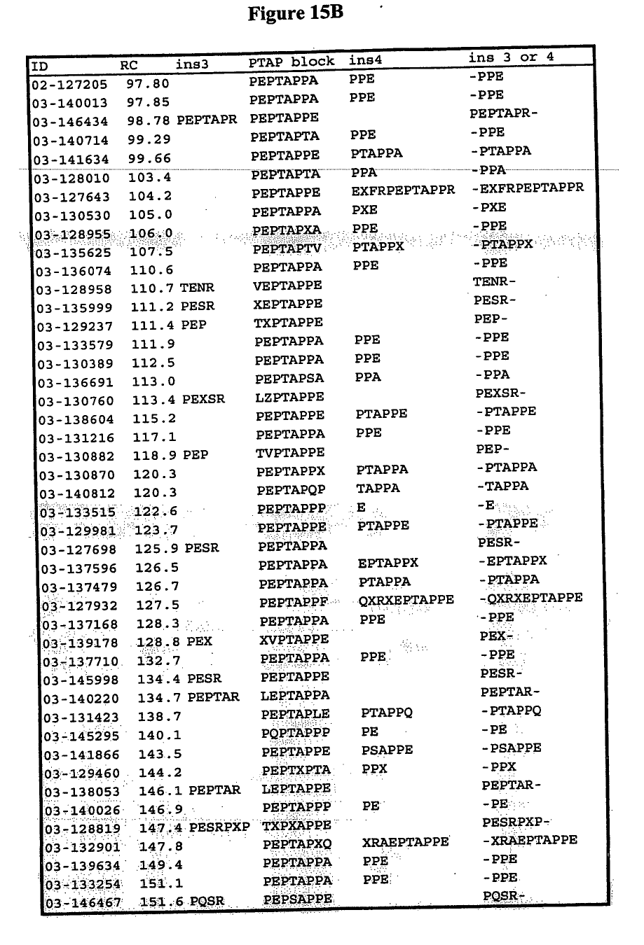

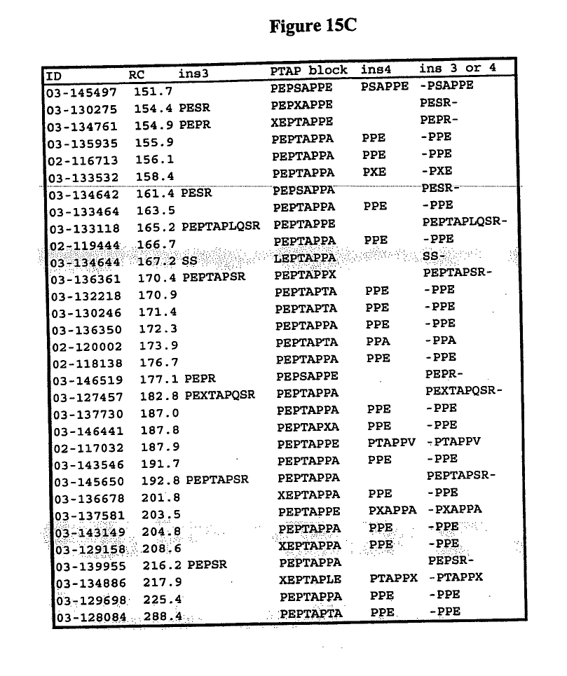

A second analysis was subsequently performed in which 544 wild-type, subtype B samples with gag, PR and RT genotype and RC values available were analyzed, spanning the entire distribution of RC. Two different RC thresholds were used, corresponding to the 10^{th} and 90^{th} percentiles of the RC distribution (54% and 180%, respectively). The analyses performed were similar to those described above, except that individual amino acid variants at each position in gag were considered separately, and all mutations were evaluated using the Student t-test. Also, a newer amino acid alignment algorithm was used which classified insertions near the PTAP domain in two categories, those which are placed on the N-terminal side (between amino acids 453 and 454) and those placed on the C-terminal side (between amino acids 460 and 461). Results are summarized in FIGS. 7A, 7B, 15A, 15B, and 15C. Many, though not all, of the mutations identified in the first analysis were also found in the second one, and some mutations from the second analysis were not found in the first. These differences may reflect the slightly different methods used or the makeup if the samples tested.

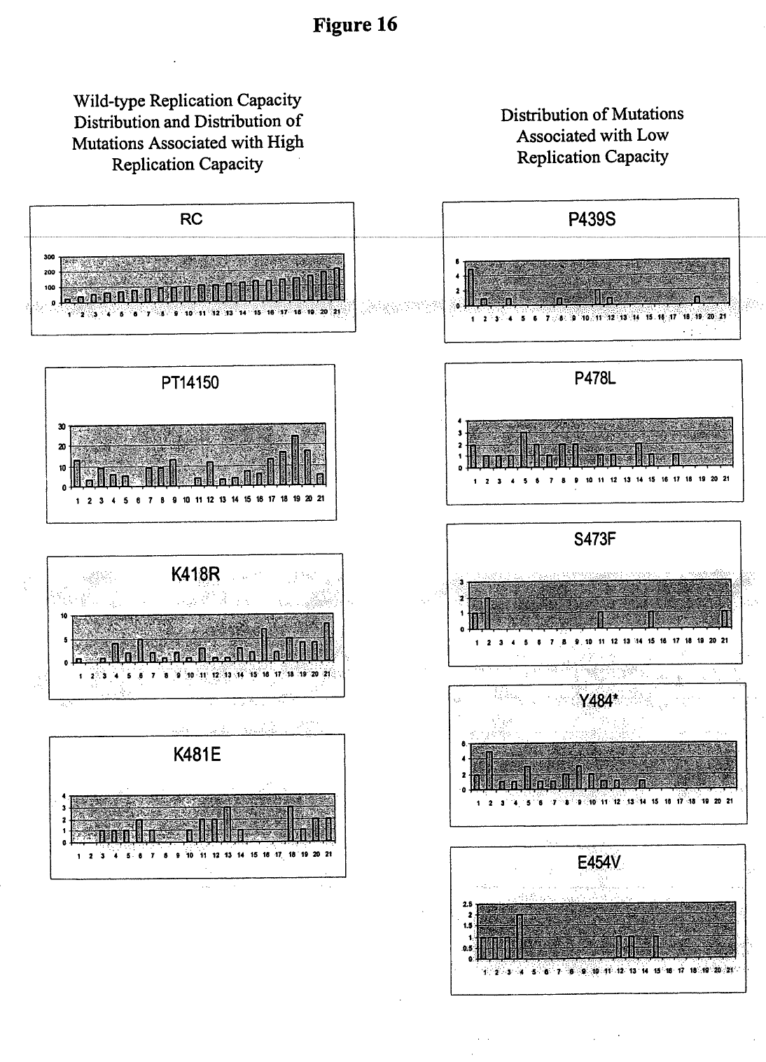

The patterns of mutation prevalence with respect to RC were also be visualized by the following procedure. Samples were sorted by increasing RC value and placed in 21 groups (“bins”). The number of samples in each bin with a given mutation is then counted and plotted as a function of bin number. Thus, mutations associated with low RC have higher prevalence in bins on the left side of the plots, whereas those associated with high RC have higher prevalence in bins on the right side. See FIG. 16.

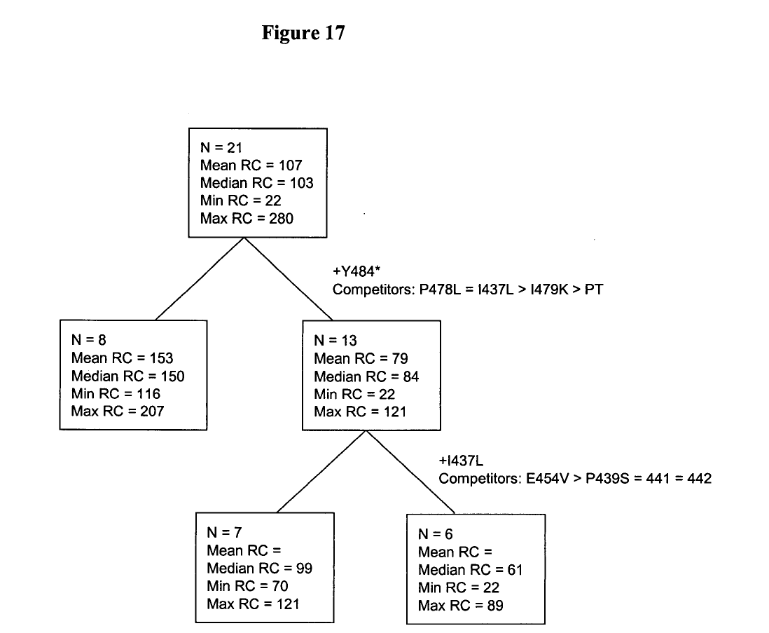

Using the mean RC in each bin from the above procedure, the prediction power of the proportion of samples with a given mutation was tested in a regression tree using CART 5.0 software (Salford Systems, San Diego Calif.). This procedure identifies the variable with the greatest ability to separate samples into two groups based on RC. For example, the presence of any mutation at position 484 in gag was the best separator variable in this analysis, followed by I437L (See FIG. 17). Competitor variables are those which are almost as strongly predictive as the best one.

Mutations Associated with Altered Replication Capacity

The experiments described above identified a number of mutations that correlate with either increased or decreased replication capacity. The specific mutations identified are presented in FIG. 6, together with statistical data showing the correlations between the mutations and altered replication capacity.

In particular, certain mutations in gag correlate with high replication capacity, while other mutations in gag correlate with reduced replication capacity. Among gag mutations that correlate with high replication capacity are certain insertion mutations between residues 458 and 459. The insertion mutations that have been identified appear to duplicate or otherwise extend the PTAP motif. This motif has recently been identified as a region of the p6 gag protein that affects the interaction of p6 gag with Tsg101, a host cellular protein. See Strack et al., 2003, Cell 114(6):689-99 and von Schwedler et al., 2003, Cell 114:701-713. The interaction between p6 gag and Tsg101 is essential to viral budding. See Strack et al., 2003, Cell 114(6):689-99. Thus, the presence of the mutation in p6 gag that duplicates the Tsg101 binding motif causes increased replication capacity. Accordingly, by identifying a mutation that affects replication capacity, an essential interaction between a viral molecule and a host cell molecule has been identified.

Important conclusions can be drawn from mutations in gag that correlate with reduced replication capacity as well. Among the mutations in gag that correlate with reduced replication capacity are mutations at codons 483, 484, and 486. Mutations in codons L483 and Y484 affect the LYP motif of the p6 gag protein, which is one of the regions of this protein that mediate an interaction with host protein AIP1. See Strack et al., 2003, Cell 114(6):689-99. The interaction between p6 gag protein and AIP1 also appears to be essential for viral budding. See id. Mutations that decrease the strength and/or efficiency of this interaction are reflected in decreased replication capacity. Thus, by identifying mutations that correlate with reduced replication capacity, and mapping the mutations to particular regions of the viral genome, the portions of viral proteins and/or nucleic acid elements that interact with host cellular proteins can be identified. Further, compounds that disrupt this interaction would be expected to be effective to reduce viral infectivity. Thus, mutations associated with reduced replication capacity can also be used to identify essential interactions between viral molecules and host cell molecules.

The figures present a number of mutations in gag, PR and RT that affect replication capacity by an as-yet unknown mechanism. Nonetheless, the regions of gag, PR and RT that comprise these mutations are important to the viral life cycle in view of the mutations' effects on replication capacity. In addition, these gag, PR and RT mutations have not previously been recognized as correlating with PI, NRTI, or NNRTI resistance. Thus, these mutations are likely in regions of gag, PR or RT that are not targeted by such antiviral drugs. By investigating the role of these regions of gag, PR and RT into the viral life cycle and identifying the molecules with which these regions interact, new targets for antiviral therapy may be identified.

### 6.3. Example 3

### Identifying Mutations Correlated with Altered Fitness in Drug-Naïve Patients

This example describes identification of mutations associated with altered fitness identified in a different patient cohort; while Example 2 focused on viruses identified as wild-type by genotypic criteria for resistance or susceptibility to all tested protease inhibitors or reverse transcriptase inhibitors, this Example focuses on viruses from patients that had not been treated with any anti-viral agents.

Sample Datasets:

The dataset included 356 wild-type samples for which genotype data and RC values was available: 108 samples from the project AIEDRP (acutely infected patients); 247 samples from the project GSK 30009 (baseline samples from a trial with entry criteria including no previous antiretroviral therapy); and the sequence NL4-3 used as a reference with RC of 100%. As discussed above, none of the patients had previously been treated with an anti-HIV therapeutic agent.

Sequence Alignment:

The sequence data in the dataset included the portion of the Gag gene from nucleotide 1254 (amino acid 418) to 1500 (amino acid 500) corresponding to the region coding for the C-terminus of p7 (NC), p1 and p6. The amino acid sequences were aligned using an algorithm that uses 10 pre-defined amino acid motifs (“blocks”) to anchor the sequence alignment in the most conserved regions. The sequence segments between the conserved blocks correspond to insertion events and were not aligned. The length and the presence of the insertion after the motif PTAP were computed as one of the variables.

For each position of the alignment, the amino acid variants present in more than 1% of the sequences were considered. Each position was also tested considering all amino acids (other than the wild-type) equally (represented as “X”) The sequences were recoded as a series of binary values corresponding to presence (1) or absence (0) of the selected mutations.

RC Distribution:

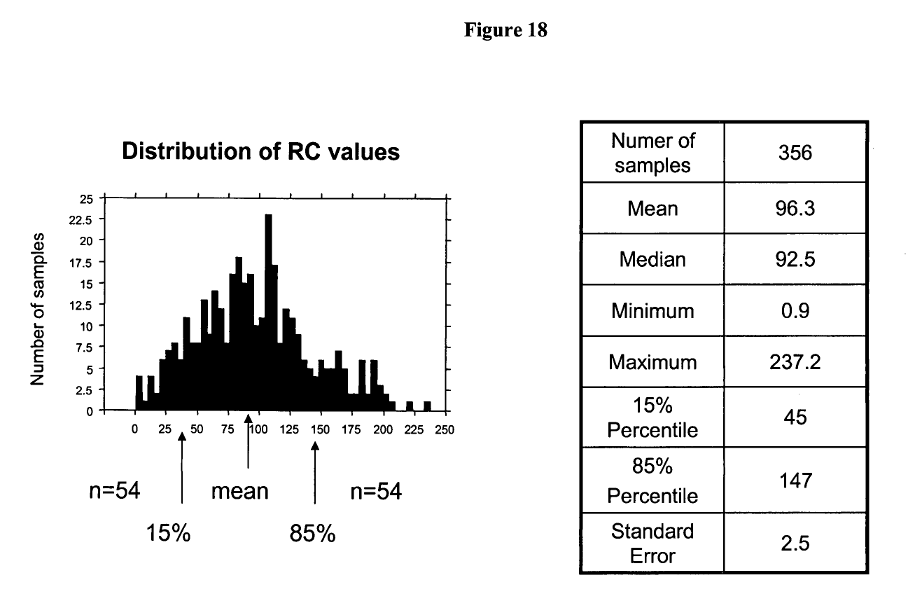

RC values were computed relative to the reference strain NL4-3 (for which RC is 100%). The median of the RC value distribution was 92.5%. The RC values corresponding to the 15% and 85% percentiles (45% and 147% respectively) were used as lower and upper cut-offs in the statistical analyses. FIG. 18 presents this data in graphical form and provides additional descriptive statistics of the dataset.

Statistical Analysis:

Two series of statistical tests were performed to evaluate the association of mutations with low RC (using the lower cut-off) and with high RC (using the upper cut-off). The association between the occurrences of the mutation with RC (recoded as lower than or greater than the cut-off) was tested separately using the Fischer's Exact test, as described above.

Results:

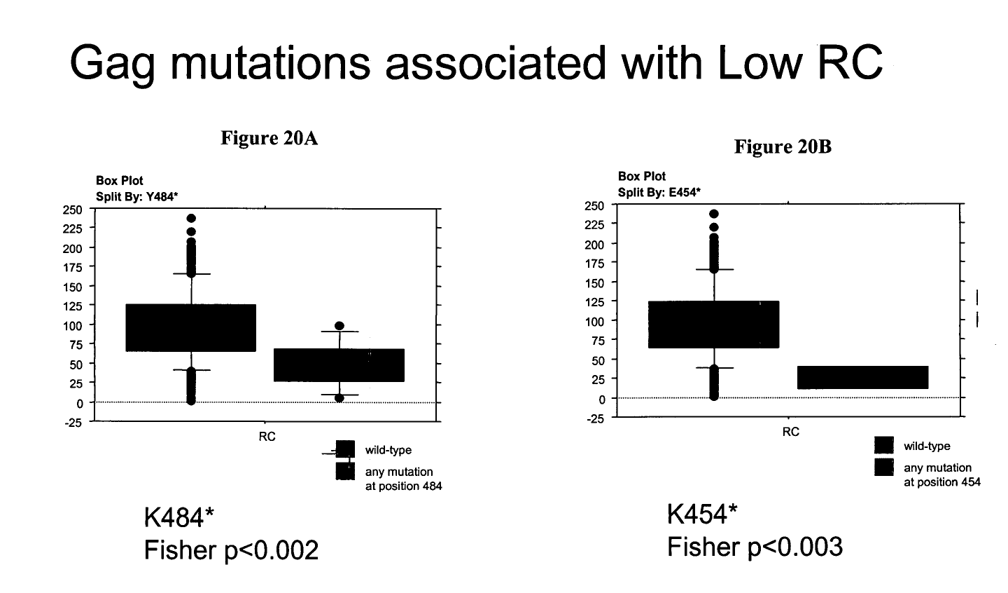

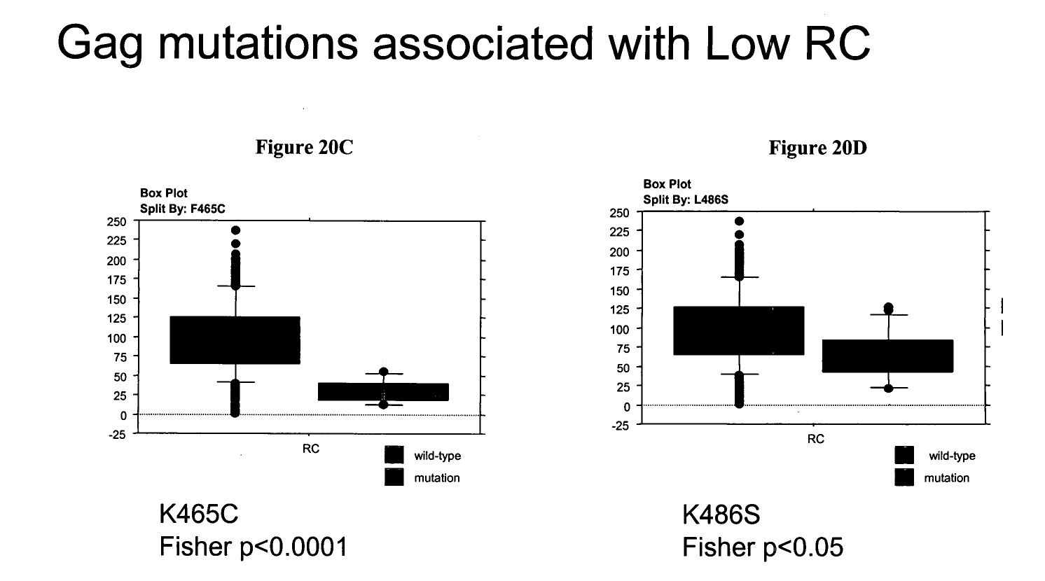

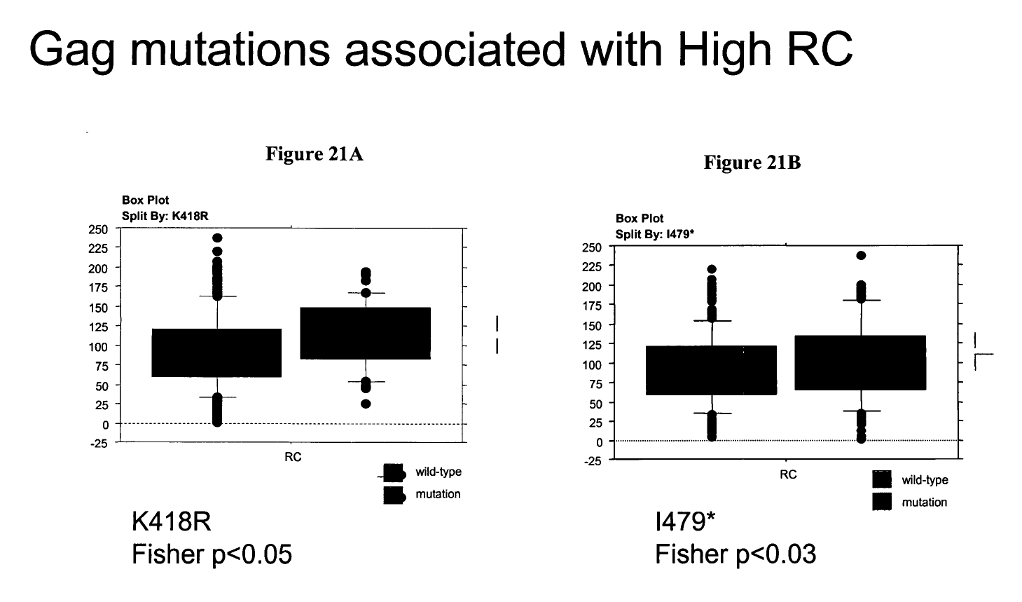

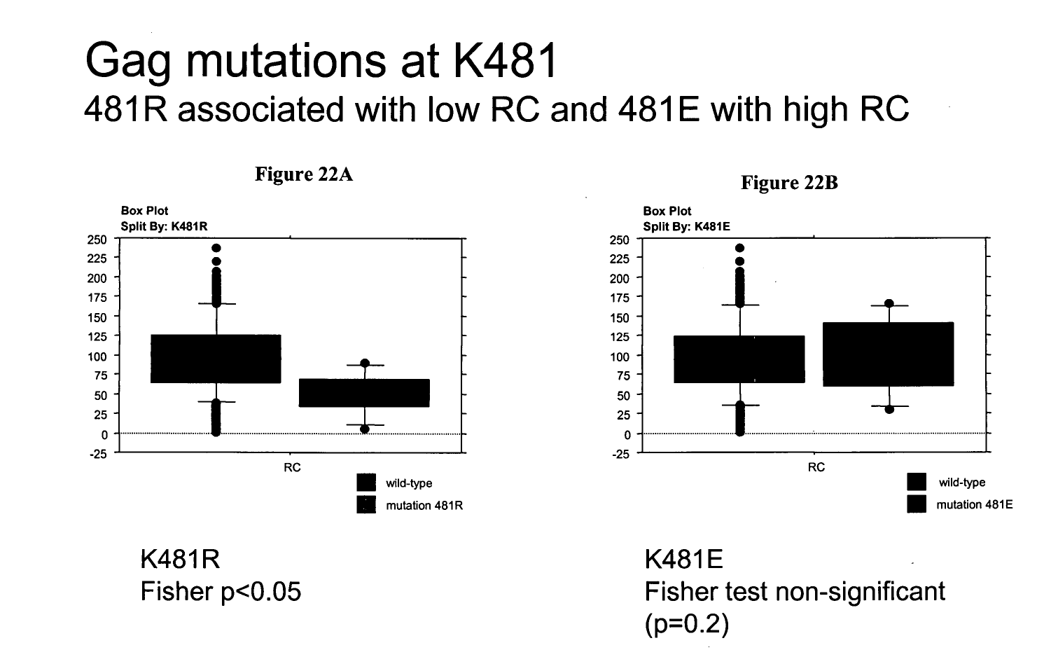

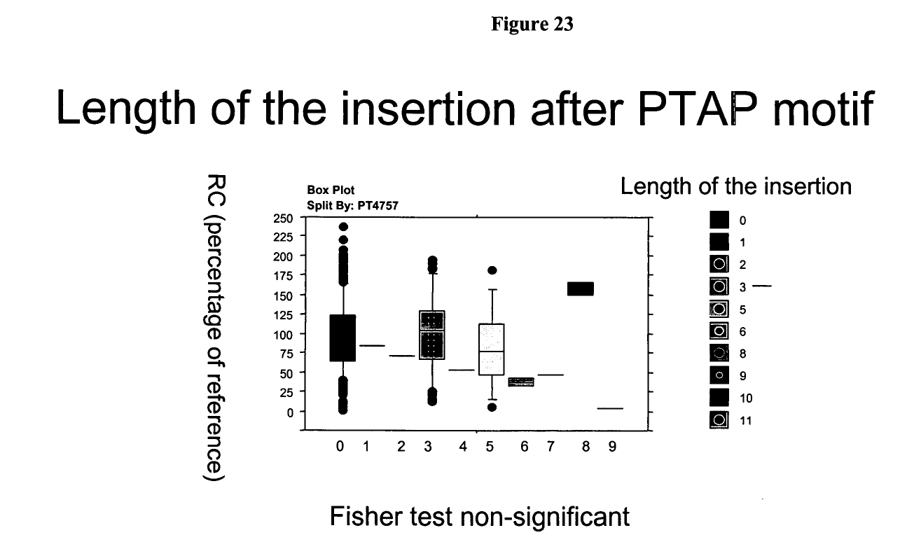

A table showing the results of the analysis is presented in FIGS. 19 and 20. In summary, the positions/mutations that were significantly associated with low RC were 484X, 454X, 465C, 481R. Box plots showing the distributions of RC observed for these mutations are presented in FIGS. 20A-D. Interestingly, a different mutation at codon 481, 481E, was associated with high RC, though the association was not significant (p=0.2), as shown in FIGS. 22A-B. The mutations that were significantly associated with high RC were 418R, 479K. Box plots showing the distributions of RC observed for these mutations are presented in FIGS. 21A-B. These mutations were among those described in the analysis of a larger dataset including wild-type samples defined by genotypic criteria as discussed above. Further, the length of the PTAP-insertion was found nearly significant for a lower RC cut-off of 50% (20% percentile of the distribution). A diagrammatic representation showing the distribution of the effects of the length of the PTAP insertion on replication capacity is presented as FIG. 23.

6.4. Example 4

### Identifying Mutations Correlated with Altered Fitness in Drug Susceptible Viruses

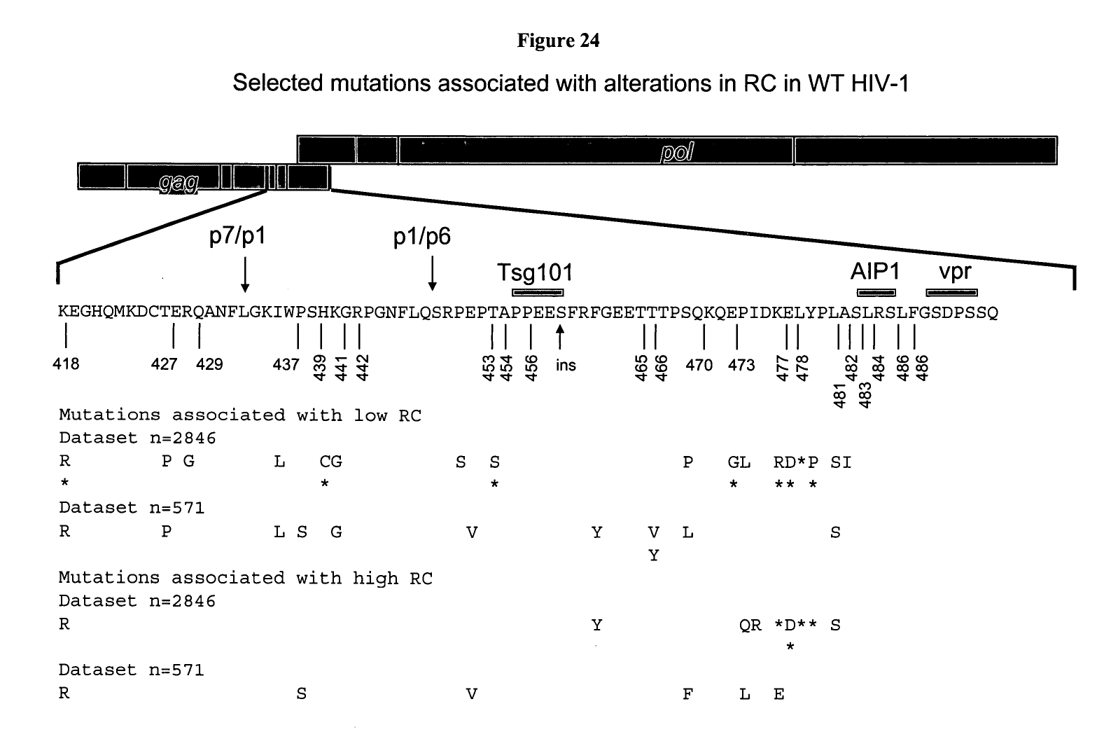

This example describes an analysis of the associations between mutations and RC values using a large dataset (n=2846 samples), and compared to those described in the previous analysis in Example 3 (n=571 samples). The dataset contained 2846 subtype B samples, wild-types as defined by genotypic criteria for all inhibitors of Protease and Reverse Transcriptase. The sequence data included the portion of the Gag gene from nucleotide 1254 (codon 418) to 1500 (codon 500) corresponding to the region coding for the C-terminal p7, p1 and p6. The amino acid sequences were aligned using an algorithm that used 10 pre-defined amino acid motifs to anchor the sequence alignment in the most conserved regions. The resulting Gag mutations, defined as the amino acids differing from the corresponding wild-type amino acids, correlating with low and high RC, determined as described in Example 3, above, were similar to those described in the previous analysis. Results are summarized in FIG. 24.

The gag mutations associated with low RC in this analysis included mutations at codon: 418, 427, 429, 437, 441, 442, 453, 456, 473, 477, 478, 481, 482, 483, 484, 486, and 487. The particular mutations or combination of mutations were K418R, T427P, R429G/K, I437L, H441C/G, K442G, P453S, T456S, S473P, E477G, P478L, K481R, E482D, Y484P, L486S, and A487I. The mutations associated with high RC in this analysis included mutations at codon: 418, 465, 478, 479, 481, 482, 483, 484, and 486. The particular mutation or combination of mutations were K418R, F465Y, P478L/Q, I479R, K481x, E482D, L483x, Y484x, and L486S.

All references cited herein are incorporated by reference in their entireties.

The examples provided herein, whether actual and prophetic, are merely embodiments of the present invention and are not intended to limit the invention in any way.

1. A method for identifying a target for antiviral therapy, said method comprising determining the replication capacity of a statistically significant number of individual viruses, the genotypes of a gene of said statistically significant number of viruses, and a correlation between said replication capacities and said genotypes of said gene, thereby identifying a target for antiviral therapy.

2. The method of claim 1, wherein said replication capacity of said viruses is determined using a phenotypic assay.

3. The method of claim 1, wherein said genotypes that are determined comprise the genotypes of an essential gene of said viruses.

4. The method of claim 1, wherein said genotypes that are determined comprise the genotypes of a nonessential gene of said viruses.

5. The method of claim 1, wherein said genotypes that are determined comprise the genotypes of two or more genes of said viruses.

6. The method of claim 1, wherein said individual viruses are retroviruses.

7. The method of claim 6, wherein said retroviruses are HIV.

8. The method of claim 7, wherein said genotypes that are determined comprise genotypes of a gene that is selected from the group consisting of gag, pol, env, tat, rev, nef, vif, vpr, and vpu.

9. The method of claim 8, wherein said genotypes that are determined comprise genotypes of gag.

10. The method of claim 9, wherein said genotypes that are determined comprise a genotype of an allele of gag that comprises a mutation, insertion, or deletion.

11. The method of claim 10, wherein said allele of gag comprises a nucleic acid that encodes a mutation at codon 418, 427, 429, 437, 439, 442, 454, 465, 466, 470, 473, 478, 482, 483, 484, or 486 of gag.

12. The method of claim 11, wherein said mutation is selected from the group consisting of K418R, T427P, I437L, P439S, K442G, E454V, F465Y, T470V, T470Y, S473F, P478L, and L486S.

13. The method of claim 11, wherein said allele of gag comprises a nucleic acid that encodes a mutation at codon 418, 439, 454, 473, 478, 481, or 484 of gag.

14. The method of claim 13, wherein said mutation is selected from the group consisting of K418R, P439S, E454V, S473F, P478L, and K481E.

15. The method of claim 10, wherein said allele of gag comprises a nucleic acid that encodes an insertion between codons 460 and 461 of gag or between codons 452 and 453 of gag.

16. The method of claim 15, wherein said insertion between codons 460 and 461 comprises an insertion of between one and twelve amino acids.

17. The method of claim 16, wherein said insertion comprises an amino acid sequence that has a formula that is X₁-X₂-X₃-X₄-X₅-X₆-X₇-X₈-X₉-X₁₀-X₁₁-X₁₂, wherein:
X₁ is selected from the group consisting of P, R, E, Q, and T;
X₂ is absent or selected from the group consisting of P, R, A, S, and T;
X₃ is absent or selected from the group consisting of E, A, F, P, T, and R;
X₄ is absent or selected from the group consisting of P, R, A, and E;
X₅ is absent or selected from the group consisting of P, A, E, and T;
X₆ is absent or selected from the group consisting of A, E, P, Q, T, and V;
X₇ is absent or selected from the group consisting of P, T, and A;
X₈ is absent or selected from the group consisting of P, T, and A;
X₉ is absent or selected from the group consisting of P and A;
X₁₀ is absent or selected from the group consisting of P and E;
X₁₁ is absent or selected from the group consisting of P and E;
X₁₂ is absent or R.

18. The method of claim 17, wherein said insertion comprises an amino acid sequence that is selected from the group of E, PE, PPE, PPA, TAPPA, PTAPPA, PTAPPE, EPTAPP, PTAPPQ, PSAPPE, PTAPPV, and RPEPTAPPA.

19. The method of claim 15, wherein said insertion between codons 452 and 453 comprises an insertion of between two and ten amino acids.

20. The method of claim 19, wherein said insertion comprises an amino acid sequence that has a formula that is X₁-X₂-X₃-X₄-X₅-X₆-X₇-X₈-X₉-X₁₀, wherein:
X₁ is selected from the group consisting of P, S, and T;
X₂ is selected from the group consisting of R, D, E, Q, and S;
X₃ is absent or selected from the group consisting of P, S, Q, and N;
X₄ is absent or selected from the group consisting of R, Q, T, and S;
X₅ is absent or selected from the group consisting of P, A, R, and S;
X₆ is absent or selected from the group consisting of R and P;
X₇ is absent or selected from the group consisting of R, P, L, S, and Q
X₈ is absent or selected from the group consisting of Q, R, and S;
X₉ is absent or selected from the group consisting of S and R; and
X₁₀ is absent or R.

21. The method of claim 20, wherein said insertion comprises an amino acid sequence that is selected from the group consisting of SR, SS, PEP, PESR, PEPR, PQSR, TENR, PDQSR, PEPSR, PEQSR, PEPSAR, PEPQSR, PQPTAP, PEPTAR, PEPTAPR, PEPTAPSR and PEPTAPLQSR.

22. The method of claim 10, wherein said allele of gag comprises an insertion between codons 458 and 459 of gag.

23. The method of claim 22, wherein said insertion between codons 458 and 459 comprises an insertion of between three and fourteen amino acids.

24. The method of claim 23, wherein said insertion comprises an amino acid sequence that has a formula that is X₁-X₂-X₃-X₄-X₅-X₆, wherein:
X₁ is absent or selected from the group consisting of P and T;
X₂ is absent or E;
X₃ is absent or P;
X₄ is selected from the group consisting of P, S, and T;
X₅ is A; and
X₆ is P.

25. The method of claim 24, wherein said insertion comprises an amino acid sequence that is selected from the group of PEPSAP, TEPTAP, PEPTAP, EPTAP, PXAP, PAP, SAP, and TAP.

26. The method of claim 8, wherein said genotypes that are determined comprise genotypes of pol.

27. The method of claim 26, wherein said genotypes that are determined comprise a genotype of an allele of pol that comprises a mutation, insertion, or deletion.

28. The method of claim 27, wherein said allele of pol comprises a mutation in the region of pol that encodes protease.

29. The method of claim 28, wherein said mutation is selected from the group consisting of mutations at codons 10, 14, 15, 20, 36, 37, 39, 61, 63, 64, 71, 72, 77, and 93 of protease.

30. The method of claim 29, wherein said mutation is selected from the group consisting of I15V, K20M, M36L, N37D, P39Q, P39S, Q61N, A71T, and V77I.

31. The method of claim 27, wherein said allele of pol comprises a mutation in the region of pol that encodes reverse transcriptase.

32. The method of claim 31, wherein said mutation is selected from the group consisting of mutations at codons 39, 121, 135, 138, 196, 203, 204, 207, 210, 211, 245, 248, 275, 276, and 286.

33. The method of claim 32, wherein said mutation is selected from the group consisting of D121Y, I135V, E138A, G196E, E203D, E204D, E204K, Q207E, R211Q, V245E, E248D, K275Q, V276T, and T286P.

34. The method of claim 7, wherein said genotypes that are determined comprise genotypes of a 5′ or 3′ untranslated region.

35. The method of claim 7, wherein said at least one target that is identified comprises a nucleic acid that encodes a portion of gag, pol, env, tat, rev, nef, vif, vpr, and vpu.

36. The method of claim 7, wherein said at least one target that is identified is a nucleic acid that comprises a portion of a 5′ or 3′ untranslated region.

37. The method of claim 7, wherein said at least one target that is identified comprises a portion of a viral protein that interacts with a host cell protein.

38. The method of claim 7, wherein said at least one target that is identified comprises a portion of a first viral protein that interacts with a second viral protein.

39. The method of claim 38, wherein said first viral protein is the same protein as the second viral protein.

40. The method of claim 7, wherein said at least one target that is identified comprises a portion of a protein that is selected from the group consisting of p1 gag protein, p2 gag protein, p6* pol protein, p6 gag protein, p7 nucleocapsid protein, p17 matrix protein, p24 capsid protein, p55 gag protein, p10 protease, p66 reverse transcriptase/RNAse H, p51 reverse transcriptase, p32 integrase, gp120 envelope glycoprotein, gp41 glycoprotein, p23 vif protein, p15 vpr protein, p14 tat protein, p19 rev protein, p27 nef protein, p16 vpu protein, and p12-16 vpx protein.

41. The method of claim 40, wherein said at least one target that is identified comprises a portion of gag.

42. The method of claim 41, wherein said portion of gag comprises a PTAP motif.

43. The method of claim 42, wherein said PTAP motif is at positions 455-458 of gag.

44. The method of claim 41, wherein said portion of gag comprises a LYP or KQE motif.

45. The method of claim 41, wherein said portion of gag comprises an amino acid that is selected from the group consisting of residues 418, 427, 429, 437, 439, 442, 454, 465, 466, 470, 473, 478, 482, 483, 484, and 486.

46. The method of claim 45, wherein said portion of gag comprises residue 484 of gag.

47. The method of claim 40, wherein said at least one target that is identified comprises a portion of protease.

48. The method of claim 47, wherein said portion of protease comprises an amino acid selected from the group consisting of residues 10, 14, 15, 20, 36, 37, 39, 61, 63, 64, 71, 72, 77, and 93 of protease.

49. The method of claim 40, wherein said at least one target that is identified comprises a portion of reverse transcriptase.

50. The method of claim 49, wherein said portion of reverse transcriptase comprises an amino acid that is selected from the group consisting of 39, 121, 135, 138, 196, 203, 204, 207, 210, 211, 245, 248, 275, 276, and 286 of reverse transcriptase.

51. A method for determining that an HIV has a low replication capacity, said method comprising detecting a mutation in a codon of gag that is selected from the group consisting of 437, 439, 441, 442, 454, 478, 479, and 484.

52. The method of claim 51, wherein said mutation is selected from the group consisting of I437L, P439S, E454V, P478L, and I479K.

53. A method for determining that an HIV has an altered replication capacity, comprising detecting a mutation in a codon of gag that is selected from the group consisting of 418, 456, 456, 453, 418, 483, 481, 465, 429, 484, 481, 483, 484, 465, 454, 442, 479, 418, 479, and 486.

54. The method of claim 53, wherein the replication capacity of the HIV is increased.

55. The method of claim 53, wherein the replication capacity of the HIV is decreased.

56. The method of claim 53, wherein the mutation in gag is K418R, T456X, T456S, P453X, K418X, L483X, K481X, F465X, R429X, Y484X, K481R, L483-, Y484-, F465C, E454X, K442X, I479K, K418R, I479X, or L486S.
